(19) United States
(12) Patent Application Publication
(10) Pub. No.: US 2025/0299826 A1
(43) Pub. Date: Sep. 25, 2025

(54) OPTIMAL TUMOR MICROENVIRONMENT NORMALIZATION THERAPY

(71) Applicant: University of Connecticut, Farmington, CT (US)

(72) Inventors: Matthew David Stuber, Cambridge, MA (US); Chenyu Wang, Vernon, CT (US); Samuel Degnan-Morgenstern, Wallingford, CT (US); John Martin, Las Vegas, NV (US)

(21) Appl. No.: 18/862,378

(22) PCT Filed: May 2, 2023
(86) PCT No.: PCT/US2023/020684

§ 371 Date: Nov. 1, 2024

Related U.S. Application Data
(60) Provisional application No. 63/337,334, filed on May 2, 2022.

(51) Int. Cl. G16H 50/30; G16H 20/10; G16H 50/50

(52) U.S. Cl. CPC G16H 50/30; G16H 20/10; G16H 50/50

(57) ABSTRACT

A personalized method of treating a cancer patient with a tumor utilizing a computing system having a processing system and a memory system storing instructions that are executed by the processing system, the method having the computing system performing steps of: performing parameter estimation to determine physiological parameters π of the tumor, including vascular hydraulic conductivity and interstitial hydraulic conductivity: determining whether the selected tumor transport model is valid or invalid by solving for physiological parameters π, and upon determining that the selected tumor transport model is valid. the method includes determining a treatment: and according to the method, the treatment is applied to a cancer patient.

### CROSS-REFERENCE TO RELATED APPLICATIONS

This application claims priority to U.S. Provisional Application 63/337,334 filed on May 2, 2022, which is incorporated herein by reference in its entirety.

### STATEMENT REGARDING FEDERALLY SPONSORED RESEARCH AND DEVELOPMENT

This invention was made with government support under Award No.: 1932723 awarded by National Science Foundation. The government has certain rights in the invention.

### FIELD OF THE DISCLOSURE

The present disclosure is related to tumor therapy and more specifically to optimizing tumor microenvironment normalization therapy.

### BACKGROUND

Solid tumors feature pathophysiological abnormalities that are biophysical barriers to the transport of anticancer drugs. These barriers impede the effectiveness of such therapies by limiting their accumulation and spatial distribution. Ameliorating the pathophysiology such that tumor microenvironment (TME) components have a more “normalized” phenotype increases small-molecule and nanocarrier-based therapies' delivery and efficacy in cancer patient. However, TME normalization combined with anticancer therapies has yet to lead to cures throughout a cancer patient population. Thus, a deeper understanding of how TME normalization affects the transport of therapies within tumors is necessary to fully bypass these spatially and temporally heterogeneous biophysical barriers.

Described herein is a modeling method that can be used to construct a robust framework for determining how the normalized TME modulates biophysical barriers to transport phenomena in tumors, thereby enabling the discovery of deeper insights into effective TME normalization.

### BRIEF SUMMARY

In one aspect, a personalized method of treating a cancer patient with a tumor utilizing a computing system having a processing system and a memory system storing instructions that are executed by the processing system, the method having the computing system performing steps of: performing parameter estimation to determine physiological parameters π of the tumor, including vascular hydraulic conductivity and interstitial hydraulic conductivity; determining whether the selected tumor transport model is valid or invalid by solving for physiological parameters π, and upon determining that the selected tumor transport model is valid, the method includes determining a treatment; and according to the method, the treatment is applied to a cancer patient.

Another aspect is a computerized system comprising: a processing system and a memory system storing instructions that are executed by the processing system such that the system is configured to performing steps of: performing parameter estimation to determine physiological parameters π of the tumor, including vascular hydraulic conductivity and interstitial hydraulic conductivity; determining whether the selected tumor transport model is valid or invalid by solving for physiological parameters π. and upon determining that the selected tumor transport model is valid, the method includes determining a treatment; and the method further includes: applying the treatment to a cancer patient.

Another aspect includes a computer program product comprising a memory device having computer executable instructions stored thereon, which when executed by one or more processors cause the one or more processors to perform a plurality of operations comprising: performing parameter estimation to determine physiological parameters π of the tumor, including vascular hydraulic conductivity and interstitial hydraulic conductivity; determining whether the selected tumor transport model is valid or invalid by solving for physiological parameters π, and upon determining that the selected tumor transport model is valid, the method includes determining a treatment; and the method further includes: applying the treatment to a cancer patient.

### BRIEF DESCRIPTION OF THE DRAWINGS

FIG. 1A is a flowchart is illustrated demonstrating a systematical framework for optimal therapy design within the context of tumor microenvironment (TME) normalization.

FIG. 1B is another flowchart showing an overview of the flowchart of FIG. 1A.

FIG. 1C is a flowchart showing additional aspects related to parameter estimation identified in FIGS. 1A and 1B.

FIG. 1D is another flowchart showing additional aspects related to steps performed when determining the physiological parameters π determined during the disclosed process are deemed invalid.

FIG. 1E is another flowchart showing additional aspects related to determining a dose selection or drug size, identified in FIG. 1A.

FIG. 1F is another flowchart showing additional aspects related to simultaneously determining a dose selection or drug size, identified in FIG. 1A.

FIGS. 2A and 2B show numerical solutions and bounding results for the tumor transport model are plotted.

FIG. 3 shows a fully connected feed-forward multilayer perceptron artificial neural network surrogate model is illustrated and represents the model architecture used for the simplified parameter estimation problems considered herein.

FIGS. 4A and 4B show experimental data and corresponding regression models for (7) and (8) are respectively plotted in (4A) Lₚ versus dose; (4B) K versus dose of dexamethasone.

FIGS. 5A and 5B show radial dose-dependent interstitial fluid pressure and velocity profiles.

FIGS. 6A-6D show radial and temporal dose-dependent solute concentration profiles.

FIG. 6E shows the radial interstitial concentration profiles.

FIG. 6F shows the percentages of the spatially-averaged concentrations.

FIGS. 7A-7D show dose-dependent transvascular convective and diffusive flux profiles.

FIGS. 7E and 7F show the contributions from convective and diffusive flux to spatially-averaged concentrations versus time.

FIGS. 7G-7J show the transvascular flux profiles over the dimensionless radius

[symbol]

FIG. 8 shows cross sections of the spherical tumor 800 are illustrated in this schematic for the control case 805 (left) and 3 mg/kg DEX treatment case 810 (right).

FIGS. 9A-9D show interstitial concentrations ĉ of different sizes nanocarriers.

FIG. 10 shows a diagram of the tumor microenvironment illustrating fluid and solute transport from the blood vessels to the interstitium with high transvascular permeability.

FIG. 11 shows a system for implementing the disclosed embodiments.

FIG. 12 shows additional and/or alternative details of a system for implementing the disclosed embodiments.

The above-described and other features will be appreciated and understood by those skilled in the art from the following detailed description, drawings, and appended claims.

### DETAILED DESCRIPTION

Nanoscale anticancer therapies on the order of dozens of nanometers, including macromolecules such as polymeric micelles and antibodies, benefit from longer systemic circulation due to slower clearance, selective accumulation in tumors due to leaky tumor blood vessels, and long retention in tumor tissue due to dense fibrosis and non-functional lymphatics in the TME. In fact, nanoscale therapies are currently in use today with cancer patients. Nonetheless, leaky blood vessels, dense fibrosis, and nonfunctional lymphatics collaborate to construct biophysical barriers that reduce the effectiveness of cancer treatment. Nanoscale therapies are affected in a size-dependent manner. In tumors, plasma from circulation excessively extravasates from leaky blood vessels to the interstitial (i.e., extravascular) space, yet moves slowly because dense fibrosis limits fluid movement. Ultimately, fluid cannot be cleared because tumor lymphatics are non-functional. Thus, one distinguishing feature of tumors is an elevated interstitial fluid pressure (IFP), that eliminates transvascular convective transport of drugs in tumors by reducing the transvascular pressure gradient to zero.

Vascular normalization involves fortifying leaky tumor blood vessels by blocking angiogenesis. ECM normalization involves reversing dense fibrosis by reprogramming cancer-associated fibroblasts to a quiescent phenotype so that the fibroblasts stop producing and maintaining excessive levels of extracellular matrix (ECM). As a result, the dense fibrosis. which slows interstitial fluid movement and compresses intratumor lymphatic tumor vessels such that they are nonfunctional, is diminished. Already, vascular normalization is used with nanomedicine in patients. while ECM normalization recently succeeded in a clinical trial with small-molecule chemotherapy. “TME normalization” is an umbrella term for either or both ECM normalization and vascular normalization.

Dexamethasone, which is a glucocorticoid steroid often used to manage chemotherapy-related toxicities, can induce vascular and ECM normalization simultaneously if used at an appropriate dose and schedule. Yet, how dexamethasone affects blood vessel leakiness. fibrosis, and lymphatic vessel function towards alleviating IFP and restoring a transvascular pressure gradient is multi-factored. Each factor depends on the dose of dexamethasone differently. Furthermore, how the size of nanocarrier-based anticancer drugs interacts with these factors is unclear. Therefore, enhancing the delivery of nanocarriers is a multi-faceted engineering problem, so a model-based systems engineering approach will provide understanding to the underlying physical phenomena and complex relationships of the biological system. Throughout this disclosure, the term “nanocarrier” includes nanoscale (nm) biomolecular and macromolecular therapies, in general. It is to be appreciated that the disclosure is not limited to dexamethasone. There are many drugs or combination of drugs that achieve vascular normalization or ECM normalization or both.

Transport of nanocarriers from the systemic circulation to cancer cells includes three steps: flow through blood vessels to different regions of the tumor, transvascular transport, and transport through the interstitial space of the tumor. Specifically, the capillary vasculature is a highly dynamic region for transvascular transport of medicine, nutrients, and waste materials being exchanged between the blood vessels and the interstitial space. There are two key transvascular transport mechanisms: diffusion and convection. Generally, smaller nanocarriers benefit from diffusion using concentration gradients as an additional driving force for transvascular transport, whereas larger nanocarriers must rely on convective transport using pressure gradients due to steric hindrances that make diffusion very slow. Previous studies have indicated that diffusion is the main mechanism of mass transport across the vessel wall in tumors. because of the lack of transvascular pressure gradients. However, dexamethasone affects blood vessel leakiness, fibrosis, and lymphatic function, so it could restore transvascular pressure gradients. How diffusion and convection are affected for differently sized nanocarriers is unclear. The major properties connecting diffusion and convection to the nanocarrier concentration in a tumor are S/V, K, and Lp. TME normalization refers to any drug or combination of drugs that affect any one or combination of these properties. To investigate, a first-principles-based modeling approach can quantify the important physiological parameters that govern transport in tumors.

The vascular and interstitial transport phenomena in tumors have been extensively modeled. In addition to first-principles mechanistic models. artificial intelligence (AI) has been gradually becoming a popular model-based approach in pharmacokinetics/pharmacodynamics (PKPD) studies. An efficient machine learning model simplifies computationally intensive simulations by creating mathematically simple regression models that capture input-output relationships with high accuracy. Specifically, artificial neural networks (ANNs) are powerful computational models that are capable of approximating and predicting the behavior of such complicated systems with high accuracy and efficiency for optimal therapy design within the context of TME-normalization processes. First, deterministic global optimization is proposed to solve the parameter estimation problems and provide a rigorous quantitative foundation for in silico model discrimination. Using this foundation, the relative contributions of convection and diffusion are quantified to solute transport across the vessel walls. Moreover, an optimized TME-normalizing therapy design approach is developed for dose selection that demonstrates the relationship between dexamethasone dose and the interstitial concentration of anticancer drugs in the pharmacokinetic system. Finally, this tumor transport model is utilized to determine an optimal nanomedicine size for the greatest accumulation in the tumor interstitial space. An ANN surrogate modeling approach is proposed to reduce the computational cost of solving challenging deterministic global optimization problems for model validation, dexamethasone dose selection, and anticancer nanocarrier size selection. The details of establishing and using such machine learning models within optimization-based decision-making frameworks are presented in this work.

The method described herein enhances the practicability and predictive capabilities of tumor transport models using mechanistic and data-driven model validation approaches and rigorous methods in global optimization for stronger model-based systems engineering approaches for optimal therapy design in cancer research. The information obtained through this approach aids in the development of better models and provides deeper insight into the physical behavior of molecular transport during TME normalization to guide drug development and delivery.

The invention is further illustrated by the following non-limiting examples.

### EXAMPLES

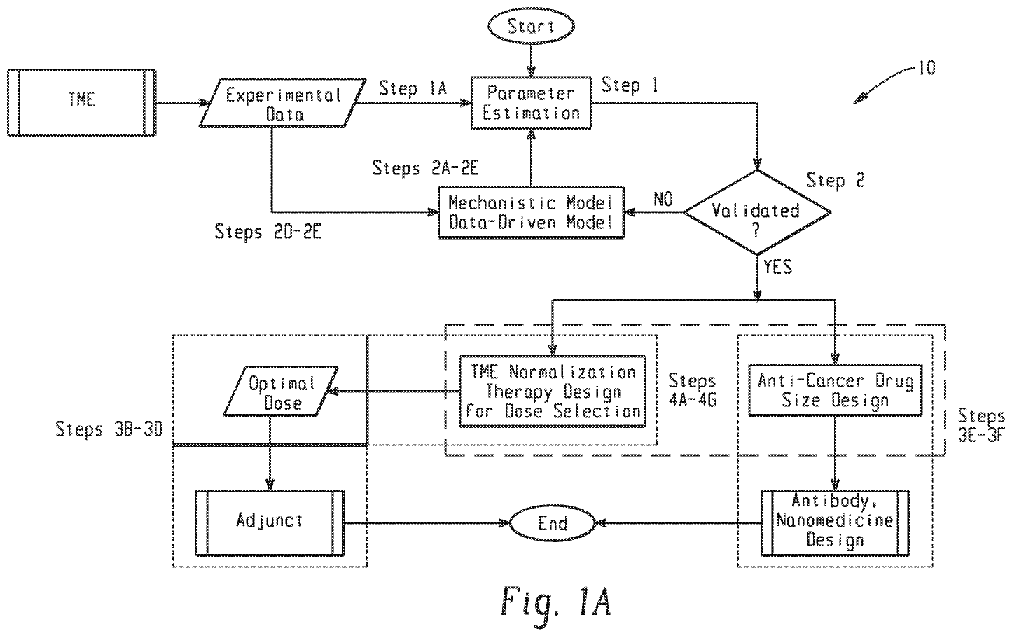

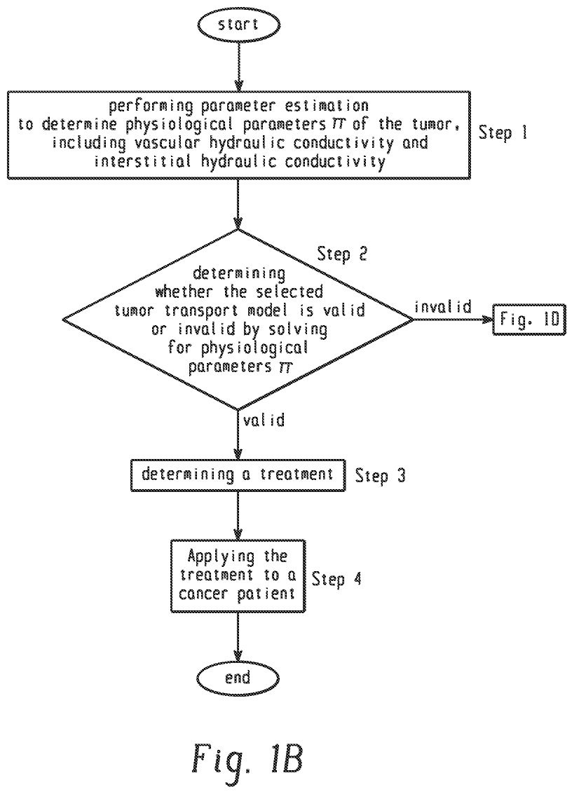

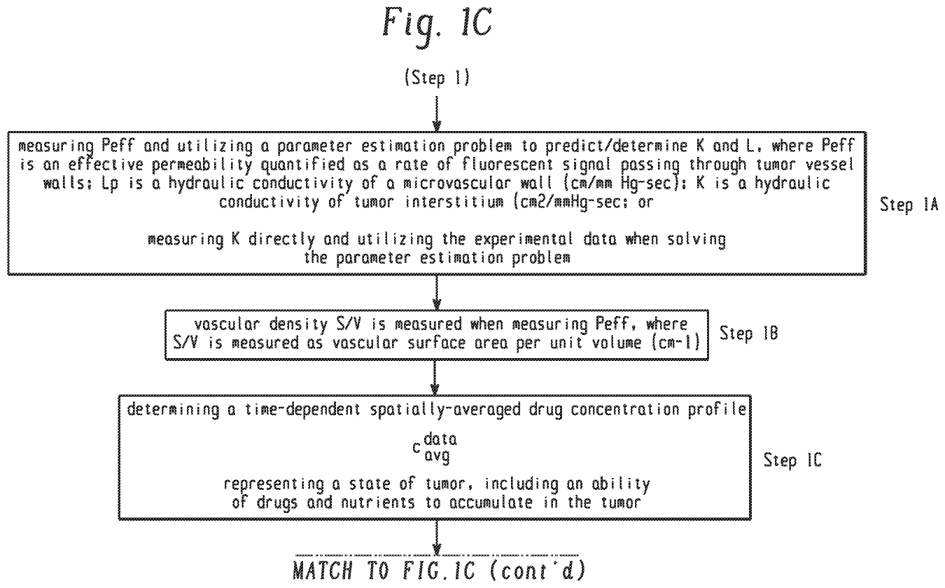

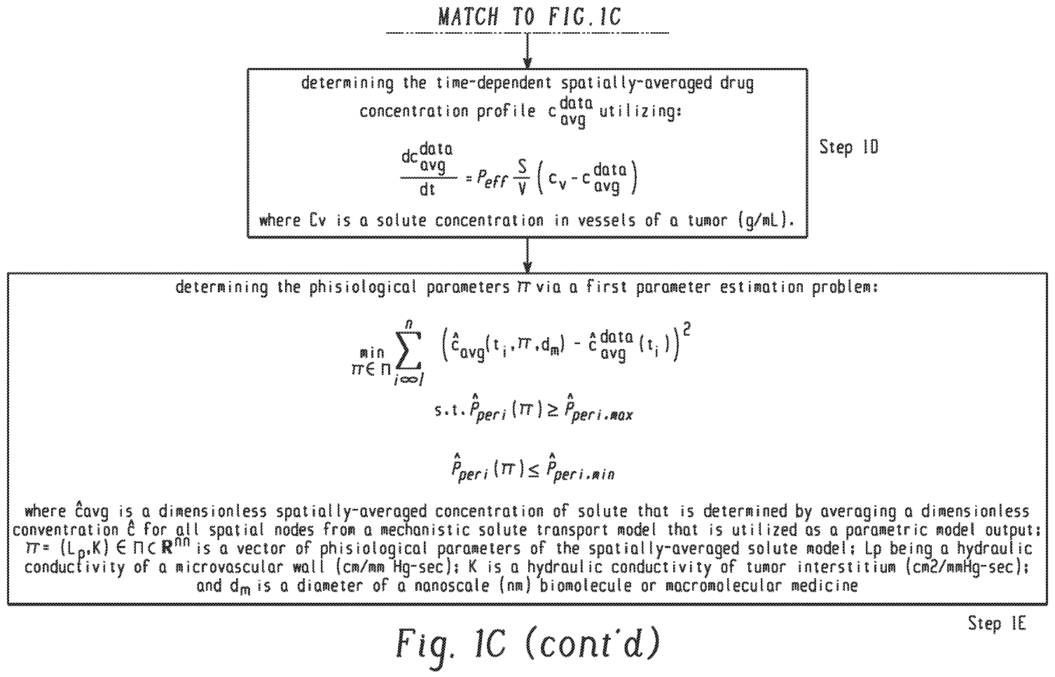

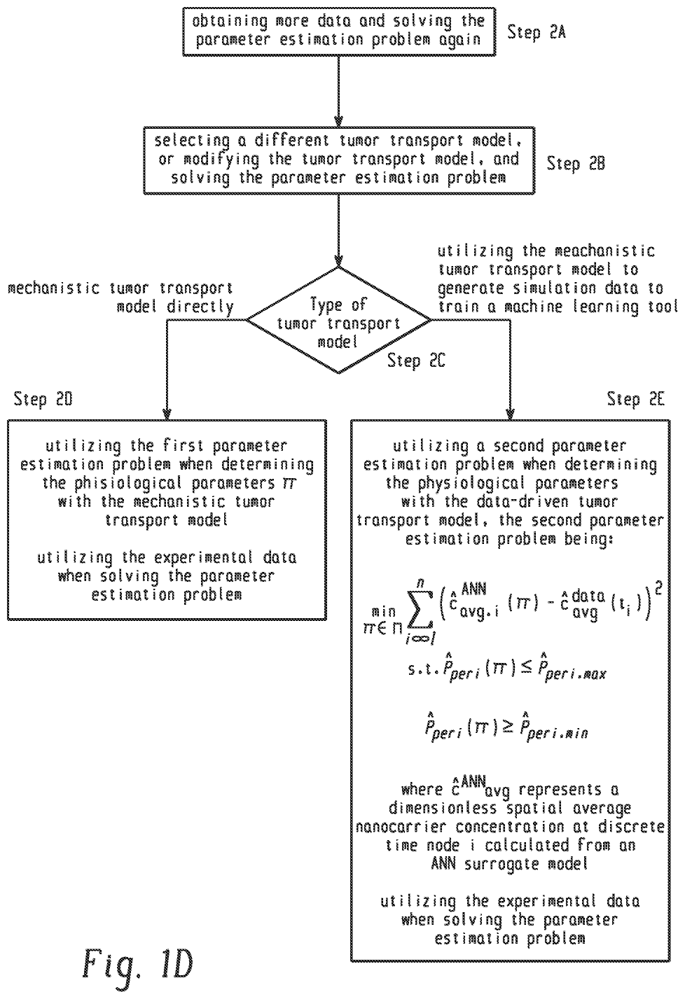

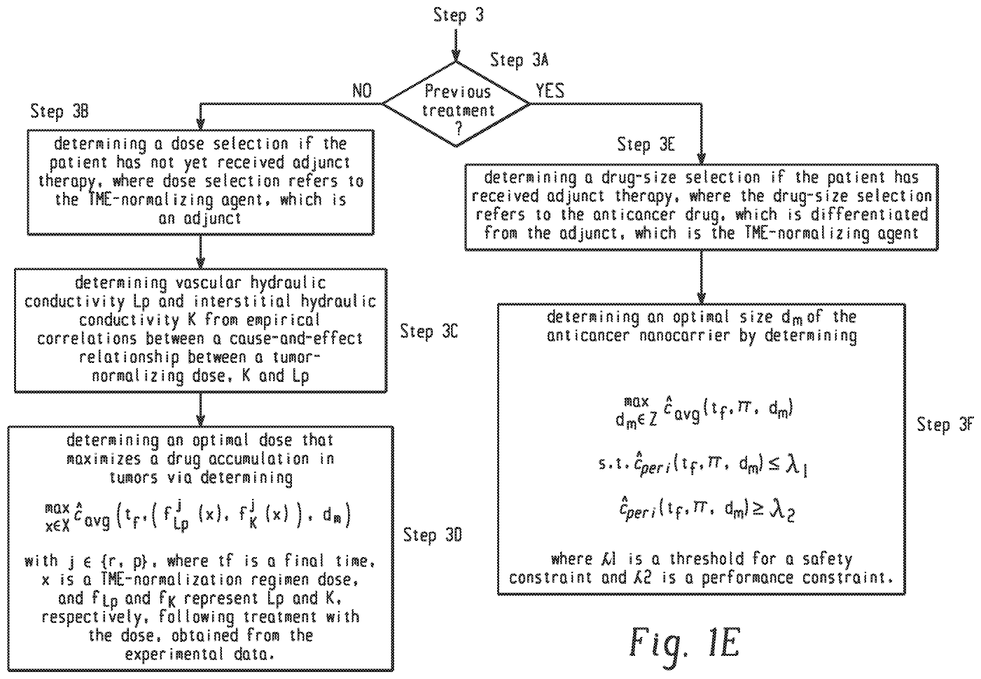

FIGS. 1A-F, discussed below, illustrate the overall systematical framework proposed for model-based TME-normalizing therapy and drug size design. To enhance the predictive capabilities of the models and provide confidence in their utility for the model-based approach for drug and therapy development. formal methods were used to estimate and quantify the critical parameters for model validation. This approach includes solving a nonconvex nonlinear program (NLP) constrained by the mechanistic tumor transport model as an unsteady partial differential equation (PDE). A simulation-based feasible path approach is proposed and the PDE-constrained optimization problem is reformulated as a box-constrained NLP. In addition, ANN machine learning methods are proposed to construct surrogate models for reducing the time costs of solving global optimization problems. Moreover, the well-established mechanistic and ANN models are also used in TME-normalizing therapy design for optimal neoadjuvant dose selection as well as drug size design for anticancer nanocarriers.

FIG. 1A is a flowchart 10 demonstrating a systematical framework for optimal therapy design within the context of tumor microenvironment (TME) normalization. Based on the experimental data, parameter estimation is utilized to validate/invalidate a proposed mechanistic model or data-driven model of the tumor. Validated models are then applied to TME normalization therapy design for dose selection and anticancer drug size design. Note that the TME normalization therapy design and drug size design in the dashed line box can be implemented separately, sequentially or simultaneously. While various blocks in FIG. 1A are discussed in greater detail below, and are addressed in FIGS. 1B-1F, the TME block represents a physical tumor with a microenvironment consisting of blood vessels, cancer cells, fibroblasts, collagen, hyaluronan, and other components of tissues, from which the disclosed process is: (i) measuring and collecting data; (ii) determining an appropriate therapy; and (iii) modifying and normalizing as part of the therapy.

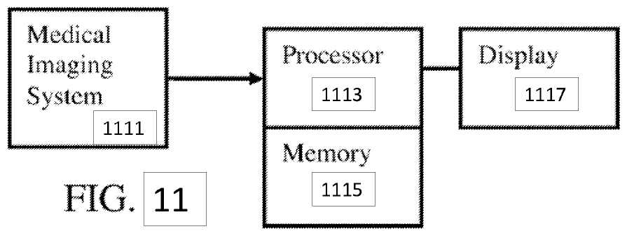

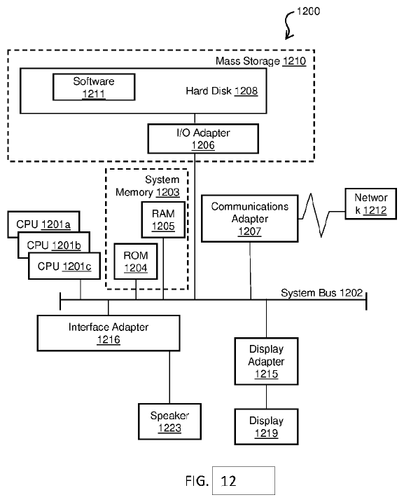

FIG. 1B is another flowchart showing an overview of the flowchart of FIG. 1A. As shown in step 1, the method includes a computing system (or generally a system), such as that illustrated in FIGS. 11 and 12, performing parameter estimation to determine physiological parameters π of the tumor, including vascular hydraulic conductivity and interstitial hydraulic conductivity. As shown in step 2, the method includes determining whether the selected tumor transport model is valid or invalid by solving for physiological parameters π. If the determination is “valid” then as shown in step 3 the method includes the system determining a treatment. As shown in step 4, the method includes a doctor (or other actor) applying the treatment to a cancer patient. If the parameters are invalid at step 2, then the process continues to FIG. 1D, discussed below.

FIG. 1C is a flowchart showing additional aspects related to parameter estimation identified in FIGS. 1A and 1B. As shown in step 1A, the method includes the system measuring Peff and utilizing a parameter estimation problem to predict/determine K and L, where Peff is an effective permeability quantified as a rate of fluorescent signal passing through tumor vessel walls; Lp is a hydraulic conductivity of a microvascular wall (cm/mm Hg-sec); K is a hydraulic conductivity of tumor interstitium (cm2/mmHg-sec); or measuring K directly and utilizing the experimental data when solving the parameter estimation problem. As shown in step 1B, vascular density S/V is measured when measuring Peff., measured as vascular surface area per unit volume (cm-1). As shown in step 1C, the method includes determining a time-dependent spatially-averaged drug concentration profile.
c_{avg}^{data}
   representing the spatial-average concentration of drug and/or nutrients in the tumor (which is time dependent). The variable may be considered as representing a state of a tumor. The term “state” is short for “state variable”, which, in this case, is concentration inside the tumor. It is a function (dependent on) the physiological properties of the tumor. As shown in step 1D. the method includes determining the time-dependent spatially-averaged drug concentration profile, utilizing:

$$\frac{d {c}_{avg}^{data}}{dt} = {P}_{eff} \frac{S}{V} ( {c}_{v} - {c}_{avg}^{data} )$$

where cᵥis a solute concentration in vessels of a tumor (g/mL).

As shown in step 1E, the method includes the system determining the physiological parameters π via a first parameter estimation problem:

$${min}_{π ∈ \prod } {\sum }_{i = 1}^{n} {( {\hat{c}}_{avg} ( {t}_{i} , π , {d}_{m} ) - {\hat{c}}_{avg}^{data} ( {t}_{i} ) )}^{2} \\ s . t . \\ {\hat{p}}_{peri} ( π ) \leq {\hat{p}}_{peri , m ax} \\ {\hat{p}}_{peri} ( π ) \geq {\hat{p}}_{peri , m i n} ,$$

where ĉ_{avg} is a dimensionless spatially-averaged concentration of solute that is determined by averaging a dimensionless concentration ĉ for all spatial nodes from a mechanistic solute transport model that is utilized as a parametric model output; π=(L_{P}, K)∈Π⊂ [symbol] ⁿ^{_{π}} is a vector of physiological parameters of the spatially-averaged solute transport model; Lp being a hydraulic conductivity of a microvascular wall (cm/mm Hg-sec); K is a hydraulic conductivity of tumor interstitium (cm²/mmHg-sec); and dm is a diameter of a nanoscale (nm) biomolecule or macromolecular medicine. The intention is to show that the disclose method works for nanocarriers as well as antibodies to include both types of emerging treatments.

FIG. 1D is another flowchart showing additional aspects related to steps performed when determining the physiological parameters π determined during the disclosed process are deemed invalid. As shown in step 2A, the method includes the system obtaining more data and solving the parameter estimation problem again. As shown in step 2B, the method includes the system selecting a different tumor transport model, or modifying the tumor transport model, and solving the parameter estimation problem. As shown in step 2C, the method includes the system making a decision based on the type of tumor transport model that is utilized. Specifically a decision is made based on whether a mechanistic tumor transport model is utilized directly, or the mechanistic tumor transport model it utilized to generate simulation data to train a machine learning model (e.g., an ANN model). If a mechanistic tumor transport model is utilized directly then at step 2D, the method includes the system utilizing the first parameter estimation problem when determining the physiological parameters π with the mechanistic tumor transport model. Otherwise, as shown in step 2E, the method includes the system utilizing a second parameter estimation problem when determining the physiological parameters π with a data-driven tumor transport model, which is less computationally burdensome compared with the mechanistic tumor transport model. The second parameter estimation problem is:

$${min}_{π ∈ \prod } {\sum }_{i = 1}^{n} {( {\hat{c}}_{avg}^{ANN} ( π ) - {\hat{c}}_{avg}^{data} ( {t}_{i} ) )}^{2} \\ s . t . \\ {\hat{p}}_{peri} ( π ) \leq {\hat{p}}_{peri , m ax} \\ {\hat{p}}_{peri} ( π ) \geq {\hat{p}}_{peri , m i n} ,$$

where ĉ^{ANN} represents a dimensionless spatial average nanocarrier concentration at discrete time node i calculated from an ANN surrogate model. The system utilizes the experimental data when solving these parameter estimation problems.

FIG. 1E is another flowchart showing additional aspects related to determining a dose selection or drug size, identified in FIG. 1A. As shown in step 3A a determination is made as to whether there is a previously applied and quantified treatment. If not, then as shown in step 3B the method includes the system determining a dose selection if the patient has not yet received adjunct therapy, where dose selection refers to the TME-normalizing agent, which is an adjunct. As shown in step 3C, the method includes the system determining vascular hydraulic conductivity Lp and interstitial hydraulic conductivity K from empirical correlations between a cause-and-effect relationship between a tumor-normalizing dose, K and Lp. As shown in step 3D, the method includes the system determining an optimal dose that maximizes a drug accumulation in tumors via determining:

$${max}_{x ∈ X} {\hat{c}}_{avg} ( {t}_{f} , ( {f}_{{L}_{p}}^{f} ( x ) , {f}_{K}^{j} ( x ) ) , {d}_{m} )$$

with j∈{r, p}, where t_{f} is a final time, x is a TME-normalization regimen dose, and f_{L,p}^{j} and fₖ^{j} represent Lₚ and K, respectively. following treatment with the drug, obtained from the experimental data. Two regression equations were considered: “rational and polynomial”. The embodiments are not limited by those types of equations, and chose these to show exactly that. If at step 3A the determination was “Yes” then at step 3E the method includes the system determining a drug-size selection if the patent has received adjunct therapy where the drug-size selection refers to the anticancer drug, which is differentiated from the adjunct, which is the TME-normalizing agent. This refers to the anticancer drug, which is differentiated from the “adjunct”, which is the TME-normalizing agent. At step 3E, the method includes the system determining an optimal size dₘ of the anticancer nanocarrier by determining

$${max}_{{d}_{m} ∈ Z} {\hat{c}}_{avg} ( {t}_{f} , π , {d}_{m} ) \\ s . t . {\hat{c}}_{peri} ( {t}_{f} , π , {d}_{m} ) \leq {λ}_{1} \\ {\hat{c}}_{peri} ( {t}_{f} , π , {d}_{m} ) \geq {λ}_{2} ,$$

where λ₁ is a threshold for a safety constraint and λ₂ is a performance constraint.

FIG. 1F is another flowchart showing additional aspects related to simultaneously determining a dose selection or drug size, identified in FIG. 1A. FIG. 1F is an alternate process compared with FIG. 1F. As shown in step 4A, the method includes the system simultaneously executing determining the dose selection module and the drug-size. As shown in step 4B the method includes the system applying empirical correlations that relate the tumor physiology to adjunct dose. As shown in step 4C the method includes the system making the same determination as in step 2C. If the mechanistic tumor transport model is used directly, then as shown in step 4D the method includes the system determining vascular hydraulic conductivity Lₚ and interstitial hydraulic conductivity K from empirical correlations between a cause-and-effect relationship between a tumor-normalizing dose, K and Lₚ. At step 4E, the method includes the system determining

$${max}_{x ∈ X , {d}_{m} ∈ Z} {\hat{c}}_{avg} ( {t}_{f} , ( {f}_{{L}_{p}}^{f} ( x ) , {f}_{K}^{j} ( x ) ) , {d}_{m} ) \\ s . t . {\hat{c}}_{peri} ( {t}_{f} , ( {f}_{{L}_{p}}^{f} ( x ) , {f}_{K}^{j} ( x ) ) , {d}_{m} ) \leq {λ}_{1} \\ {\hat{c}}_{peri} ( {t}_{f} , ( {f}_{{L}_{p}}^{f} ( x ) , {f}_{K}^{j} ( x ) ) , {d}_{m} ) \geq {λ}_{2} ,$$

with j∈{r, p}, where t_{f} is a final time, x is an anticancer drug dose, and f_{Lp}^{j} and fₖ^{j} represent Lₚ and K, respectively, following treatment with, obtained from the experimental data, λ₁ is a threshold for a safety constraint. Otherwise, at step 4F, the method includes the system determining vascular hydraulic conductivity Lₚ and interstitial hydraulic conductivity K from empirical correlations between a cause-and-effect relationship between a tumor-normalizing dose, K and Lₚ. At step 4G, the method includes the system determining

$${max}_{x ∈ X , {d}_{m} ∈ Z} {\hat{c}}_{avg}^{ANN} ( ( {f}_{{L}_{p}}^{f} ( x ) , {f}_{K}^{j} ( x ) ) , {d}_{m} ) \\ s . t . {\hat{c}}_{peri}^{ANN} ( ( {f}_{{L}_{p}}^{f} ( x ) , {f}_{K}^{j} ( x ) ) , {d}_{m} ) \leq {λ}_{1} \\ {\hat{c}}_{peri}^{ANN} ( ( {f}_{{L}_{p}}^{f} ( x ) , {f}_{K}^{j} ( x ) ) , {d}_{m} ) \geq {λ}_{2} ,$$

with j∈{r, p}, where t_{f} is a final time, x is an anticancer drug dose, and f_{Lp}^{j} and fₖ^{j} represent Lₚ and K, respectively, following treatment with, obtained from the experimental data, λ₁ is a threshold for a safety constraint and λ₂ is a performance constraint.

In sum disclosed process includes 1.) performing in vivo imaging to determine Peff and possibly S/V and direct measurement of K, 2.) Executing the process of steps 1-4, 3.) determining the dose of the TME-normalization regimen and administering it, 4.) executing the process of steps 1-4, 5.) determining an anticancer drug nanocarrier size and administer it. Alternatively, instead of step 3, the process includes simultaneously determining the dose of TME-normalization regimen and anticancer drug nanocarrier size, administering the TME-normalization regimen and anticancer nanocarrier. Alternatively, instead of step 5, the process may include repeating step 3 to determine a second dose of the TME-normalization regimen and administer it, followed by steps 4 and 5.

In sum, the disclosure provides the following high-level procedures (and permutations thereof) that are conveyed by the flowchart figures: Option 1: Imaging then determine adjunct dose then administering the dose, then imaging, then determining the anticancer drug size, then administering the anticancer drug. Option 2: Imaging, then determining the anticancer drug size, then administering the anticancer drug. Option 3: Imaging, then determining the adjunct dose and anticancer drug size, then administering therapies.

Parameter Estimation and Model Validation by Deterministic Global Optimization: The glucocorticoid steroid DEX, an agent mainly used for alleviating chemotherapy side effects, has been identified as a pre-treatment adjunct agent for normalizing metastatic tumor vessels and ECM for enhanced efficacy of drug delivery. To verify the effects of DEX on nanocarrier delivery through vascular and ECM normalization processes, the optimal solutions, of the parameter estimation problems introduced in the art by deterministic global optimization, are determined. This approach is significant because only global optimal solutions can guarantee the most accurate fit to the obtained experimental data. The mechanistic tumor transport model used in this work is introduced in the Supplementary Example 1.

Previously, a series of experiments were conducted in vivo to investigate the efficacy of DEX. In these experiments, immunocompetent mice bearing orthotopic 4T1 breast cancer were treated with 3 and 30 mg/kg DEX daily for 4 days. After which, two types of fluorescent dyes (70 kDa rhodamine-bound dextran and 500 kDa FITC-bound dextran) were injected as tracers. In vivo confocal laser scanning microscopy was employed to characterize the spatiotemporal distribution of dextrans in mouse tumors treated with different doses of DEX. Based on the intravital microscopy images, the effective permeability P_{eff} was quantified as the rate of nanoparticle fluorescent signal passing through the vessel walls normalized to the vessel surface area and the transvascular concentration difference. The effective permeability includes both convective and diffusive components; however, it significantly overestimates the diffusive part and may not be consistent with actual transcapillary transport. Then, the spatial average concentration of the interstitial space (dc ata av/dt) was calculated from the conservation equation, i.e.:

$$\frac{d {c}_{avg}^{data}}{dt} = {P}_{eff} \frac{S}{V} ( {c}_{v} - {c}_{avg}^{data} ) ,$$

where cᵥ is the solute concentration in the vessels of a tumor (g/mL) and s/v is the vascular surface area per unit volume (cm⁻¹). This serves as an experimental concentration profile for subsequent parameter estimation problems used for elucidating the physiological effects of DEX treatment.

In this work, a similar approach is taken whereby the dimensionless spatially-averaged concentration of solute c^{data}_{avg} (determined from the overall conservation equation) serves as an experimental concentration profile for each P_{eff} measured experimentally and is used for parameter estimation of the mechanistic model of interest. Deterministic global optimization methods are used to validate the mechanistic model by finding the parameter values that result in the proposed model fitting the experimental data as best as possible, and subsequently verifying the TME-normalization process. The objective function is formulated as the sum-of-squared errors (SSE) between the average concentration profile predicted by the model and the measured data (from the overall conservation expression with the experimentally measured P_{eff}) at discrete time points over the entire time horizon of the experiment. Inequality constraints are formulated for the IFP profiles based on experimentally determined values. The parameter estimation problem is formulated as:

$${min}_{π ∈ \prod } {\sum }_{i = 1}^{n} {( {\hat{c}}_{avg} ( {t}_{i} , π , {d}_{m} ) - {\hat{c}}_{avg}^{data} ( {t}_{i} ) )}^{2} \\ s . t . \\ {\hat{p}}_{peri} ( π ) \leq {\hat{p}}_{peri , m ax} \\ {\hat{p}}_{peri} ( π ) \geq {\hat{p}}_{peri , m i n} , \qquad ( 1 )$$

where the dimensionless spatially-averaged concentration of solute ĉ_{avg} is calculated by averaging the dimensionless concentration ĉ for all spatial nodes (discretization details are introduced in in the discussion, below, directed to Settings for Solving Optimization Problems) from the mechanistic solute transport model (details are introduced in the Supplementary Examples) and taken as the parametric model output for the parameter estimation problem. The decision variables π=(Lₚ, K)∈π⊂ [symbol] ⁿ^{_{π}} is the vector of physiological parameters of the model to be estimated, with Lₚ the hydraulic conductivity of the microvascular wall (cm/mm Hg-sec) and K the hydraulic conductivity of tumor interstitium (cm²/mm Hg-sec). The parameter dₘ is the diameter of the nanocarrier (nm) used in the corresponding experiment. The SSE objective fits the model-predicted profile to the experimental profile at each time node tᵢ selected within the time horizon (5 min), with I∈{1, . . . , n}. For the inequality constraints, p [symbol] ₚₑᵣᵢ is introduced as the dimensionless superficial (peripheral) IFP, which is calculated by the dimensionless IFP p [symbol] in the superficial region (in the discussion, below, directed to Settings for Solving Optimization Problems), and p [symbol] _{peri,max} and p [symbol] _{peri,min} as the physical bounds of p [symbol] ₚₑᵣᵢ, with values listed in Table 1.

In Table 1, the physical bounds on the superficial (peripheral) tumor IFP for the control, 3 mg/kg, and 30 mg/kg DEX treatment case are reported here. These values are used in the parameter estimation problems formulated as (1) to ensure that physically meaningful solutions are identified.

TABLE 1

| Dose | Control | 3 mg/kg | 30 mg/kg |
|---|---|---|---|
| p\hat{ }_(peri, [min]) (mmHg) | 4.87 | 3.02 | 1.95 |
| p\hat{ }_(peri, [max]) (mmHg) | 5.67 | 3.62 | 2.45 |

Bounding Methods for Tumor Transport Model: Deterministic global optimization can prevent erroneously invalidating mechanistic models in cases where suboptimal solutions obtained by local optimization algorithms result in poor fits. Methods for solving global optimization problems in this work rely on the branch-and-bound (BnB) framework for deterministic search. Specifically, the flexible and open-source BnB-based solver EAGO is utilized. The BnB algorithm iteratively partitions the search space into successively smaller subdomains and solves a sequence of lower-and upper-bounding subproblems on each subdomain. The algorithm converges in finitely-many iterations to an e-optimal global solution or terminates with a certificate of infeasibility by comparing the obtained bounds across nodes. The upper-bounding problems typically determine a feasible local solution (if one exists) on each subdomain. The lower-bounding problems rely on the ability to calculate rigorous global bounds on all variables and functions involved in the optimization formulation. Calculating valid lower bounds for a global optimization problem is the most challenging procedure. This is especially true for PDE systems encountered in this work, as this task amounts to constructing rigorous bounds on the spatiotemporal state solutions over the entire domain of optimization variables (i.e., the reachable set).

A method is disclosed for constructing global bounds enclosing the reachable sets of the tumor transport model. Several different bounding methods are presented and analyzed in this work to determine the most effective method for use with the tumor transport model. The fundamental approach is to use the method of lines with finite differences for spatial discretization and then differential inequalities (DI) to construct state bounds of the discretized large-scale ODE-IVP system. Apart from implementing interval arithmetic (IA) for constructing bounds, a mixed interval arithmetic/affine arithmetic (IA/AA) approach was also implemented. In addition to standard DI, a modified DI approach with interval refinement operators was also implemented for problems with prescribed bounding information known a priori. An overview of these set-valued mapping approaches is introduced in Supplementary Example 2. In summary, four bounding methods are considered for comparison: IA and DI, IA and DI with interval refinement, IA/AA and DI, and IA/AA and DI with interval refinement.

Significant nonlinearity of the models poses a major challenge to efficiently constructing tight bounds. In the tumor transport model. a problematic term that requires special consideration is the solute source term that describes the transvascular mass transport of nanocarriers:

$${ϕ}_{s} = {L}_{p} \frac{S}{V} ( {p}_{v} - p ) ( 1 - σ ) {c}_{v} + P \frac{S}{V} ( {c}_{v} - c ) \frac{Pe}{{e}^{Pe} - 1} . \qquad ( 2 )$$

Here, pᵥ is the vascular pressure (mm Hg), p is the interstitial fluid pressure (IFP) (mm Hg), σ is the solute reflection coefficient, P is the vascular permeability of the solute through the vascular wall (cm/sec), c is the solute concentration in the interstitial space of the tumor (g/mL), and Pe=Lₚ(pᵥ₋₋−p)(1−−σ)/P is the Péclet number representing the ratio of the rates of convection to diffusion across the vascular wall.

The solute source term suffers from the dependency problem of IA (i.e., the overestimation of interval operations due to the same variables being treated independently). The nonlinearity caused by the exponential terms significantly magnifies this overestimation. The dependency problem is overcome using the following strategy. Since Pe appears both in the numerator and in the denominator of the term [symbol] in (2), without special consideration, the dependency problem will lead to an appreciable overestimation of the bounds that will be detrimental to the BnB procedure. To avoid this, the function z(x)=x/(e^{x}−1) is considered, where a real interval Z=[z^{L}, z^{U}] is sought such that z(x)∈Z for every x∈[Pe^{L}, Pe^{U}], for known values Pe^{L} and Pe^{U}. It is easy to prove that z is a monotonically decreasing function of x, and therefore, the exact bounds on the range of z on the domain [Pe^{L}, Pe^{U}], can be derived as:

$${z}^{L} = \frac{{Pe}^{U}}{{e}^{{Pe}^{U}} - 1} , \\ {z}^{U} = \frac{{Pe}^{L}}{{e}^{{Pe}^{L}} - 1} .$$

The definitions of these exact bounds are used throughout this work.

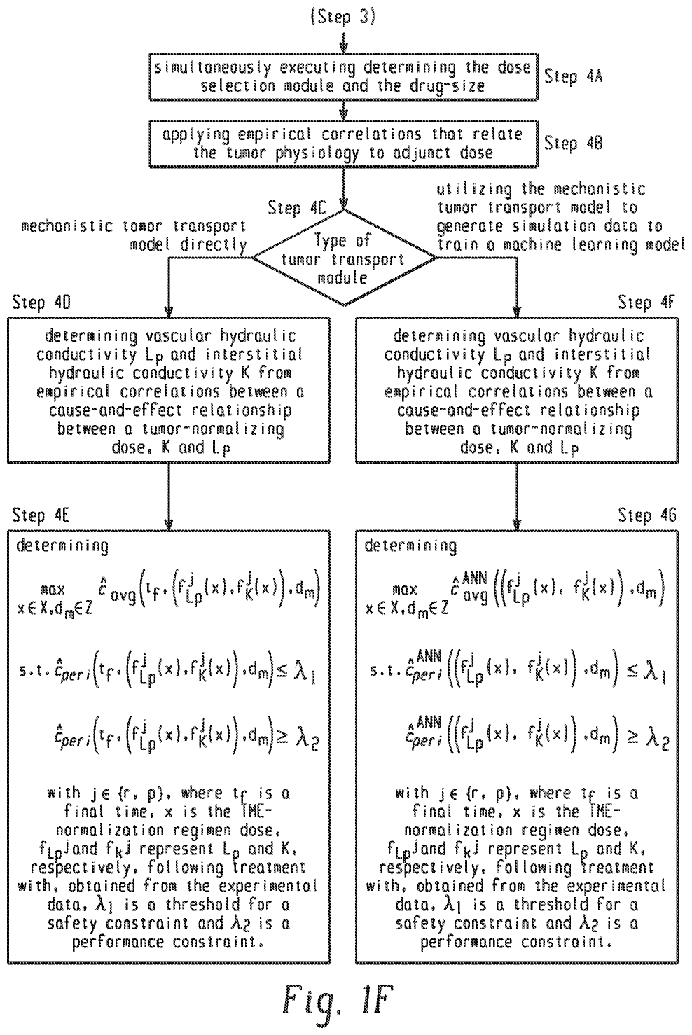

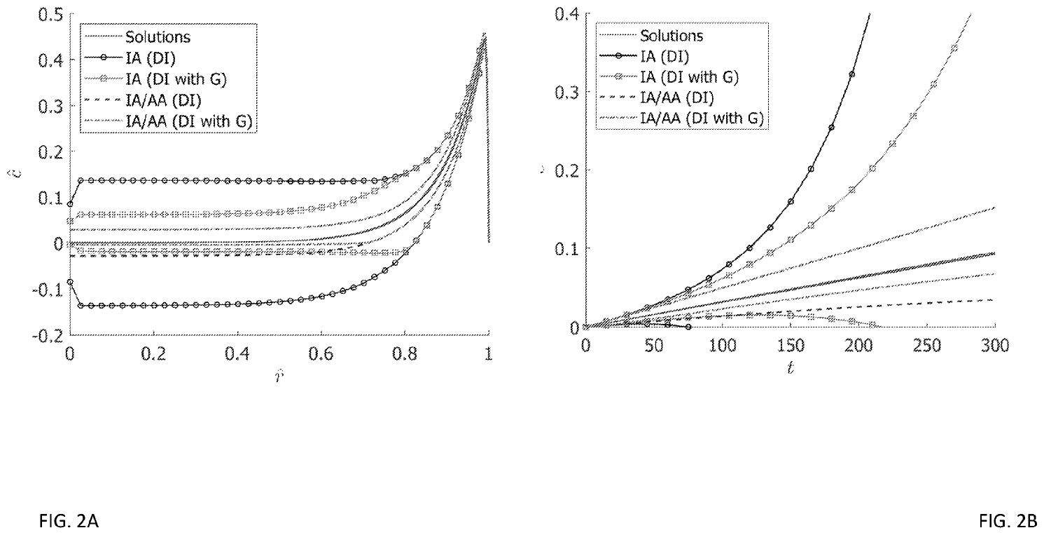

Bounds on the state variables of the tumor transport model were constructed based on four approaches. The spatial domain was discretized into N=100 nodes and the discrete-time DI scheme was used to construct the bounds through the simulation time (5 min) with 21 time steps. The two physiological parameters are considered as decision variables and bounded by an interval domain π=(Lₚ, K)∈π=[7.5×10⁻⁷, 7.6×10⁻⁷]×[1.15×10⁻⁶, 1.2×10⁻⁶]. The numerical solutions and bounding results are illustrated in FIGS. 2A and B for the four bounding methods considered.

To compare the effectiveness of the different bounding procedures, the volumes between the upper and lower bounds on the dimensionless concentration over the entire spatial and time domains are quantified along with the corresponding time costs for constructing these bounds, and listed in Table 2. It is observed that the time costs for pure IA and mixed IA/AA methods are almost the same, but the mixed IA/AA method can provide much tighter bounds. If taking the prescribed physical bounds ĉ∈G=[0, 5] into account with the modified DI method, both pure IA and mixed IA/AA methods can enhance the bounding results. However, the increased computational costs are nearly two orders-of-magnitude more than standard DI due to the curse of the dimensionality of the discretized systems. The dramatic burden in time cost using the modified DI method overshadows any improvement of the bounding results in this case. As indicated by the volumes in Table 2, the bounds constructed by mixed IA/AA and standard DI methods are already relatively efficient (91.3% tighter than the IA method, 62.4% tighter than the IA (DI with G) method, and only 37.6% larger than the IA/AA (DI with G) method), and the modified DI will not contribute much to reducing the conservatism. Therefore, the mixed IA/AA and standard DI method are utilized as the bounding routine for solving all global optimization problems.

In Table 2, the achieved volumes and time costs are reported for the different bounding methods considered herein. The IA/AA bounds are significantly tighter than the pure IA bounds (91% reduction in volume) without additional computational time. The IA/AA bounds are also nearly as tight as the IA/AA (DI with G) bounds (38% increase in volume) but with almost two order-of-magnitude less computational time.

TABLE 2

Bounding Methods

| | | | IA | IA/AA |
|---|---|---|---|---|
| | IA | IA/AA | (DI with G) | (DI with G) |
| Volume | 0.6438 | 0.0560 | 0.1489 | 0.0407 |
| Time (s) | 0.5342 | 0.5312 | 47.21 | 47.67 |

### Machine Learning Model

FIGS. 2A and 2B 2 show numerical solutions and bounding results for the tumor transport model are plotted. In FIG. 2A, the spatial profiles of the dimensionless concentration ĉin the tumor at t=150 s are plotted for several values of π along with the state bounds derived from pure IA, IA with modified DI, mixed IA/AA, and mixed IA/AA with modified DI. In FIG. 2B, the trajectories of the solute concentration ê in the tumor at the position r [symbol] =0.5 are plotted for several values of π along with the state bounds derived from pure IA, IA with modified DI, mixed IA/AA. and mixed IA/AA with modified DI. ĉ is approximated by corresponding numerical solutions calculated by the explicit Euler method, and the state bounds are calculated by the discrete-time DI method.

Machine learning regression is proposed to establish a computationally efficient artificial neural network (ANN) as a surrogate for the mechanistic tumor transport model. The established ANN models will then be used to solve the model validation parameter estimation problems as formulated in (1). This approach is proposed to analyze the relative performance and accuracy of ANN models to assess their applicability within the proposed framework for drug and therapy design, as well as broader contexts of scientific machine learning in cancer research and therapy. This was implemented in Julia version 1.6.1 running on an Intel Xeon W-2195 (18-core/32-thread) 2.3 GHz/4.3 GHz (base/turbo) CPU with 64 GB RAM running Windows 10 Pro. The inputs for the ANNs considered are the two physiological parameters Lₚ and K, discussed previously. Since DEX treatment normalizes the TME and, in turn, affects the transport phenomena in tumors, different ANN surrogate models were constructed to represent the control and DEX treated tumors for greater accuracy. Furthermore, since the experimental data varied slightly across the 70 kDa nanocarrier and 500 kDa nanocarrier experimental groups, separate ANN surrogate models were also constructed for greater accuracy within these mouse groups. Thus, four distinct ANN surrogates are considered: 70 kDa nanocarrier control case, 70 kDa nanocarrier 3 mg/kg and 30 mg/kg DEX treatment cases, 500 kDa nanocarrier control case, and 500 kDa nanocarrier 3 mg/kg and 30 mg/kg DEX treatment cases. For each case, the tumor transport model was parameterized by Lₚ and K. The discretized fluid and solute transport models were solved using the method of lines via the stiff QNDF solver in DifferentialEquations.jl for data acquisition. Then, the spatially-averaged concentrations over a discrete time horizon of 5 minutes were taken as outputs.

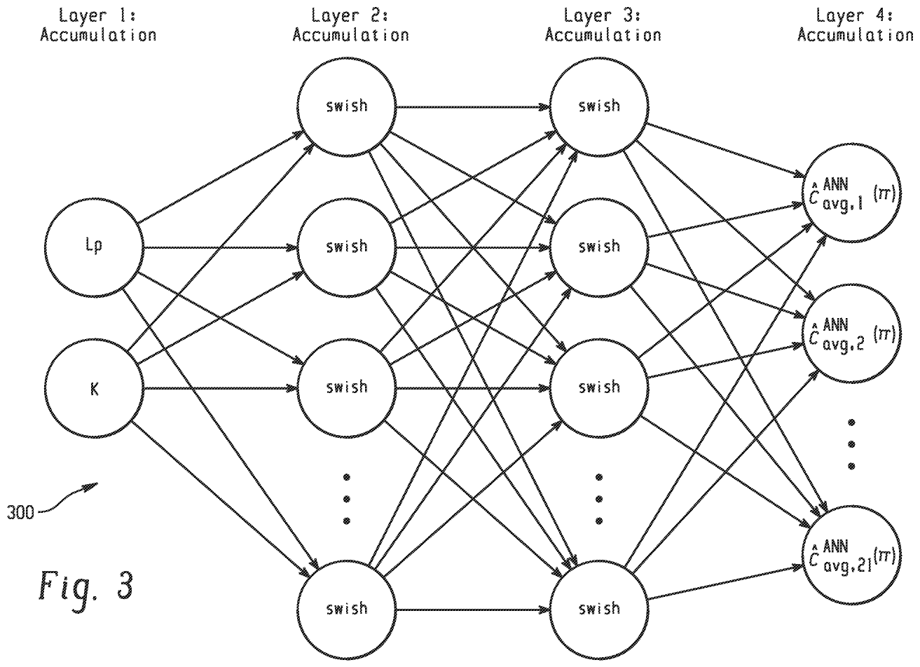

A Sobol sequence sampling protocol in Surrogates.jl was used to generate a data set of 10⁶ points within the bounds described in Table 3. The data was then scaled using min-max normalization and randomly divided into a training set (70%) and test set (30%). The ANN models were trained and constructed using Flux.jl. Architectures of 2-4 hidden layers, 16-32 nodes per hidden layer, and several different activation functions (sigmoid, tanh, gelu, and swish) were considered. Through tuning and comparison, a two-hidden-layer model with 24 neurons each with the swish activation function was chosen for use in this work. This ANN model is depicted in FIG. 3.

FIG. 3 shows a fully connected feed-forward multilayer perceptron artificial neural network surrogate model 300 and represents the model architecture used for the simplified parameter estimation problems considered herein. The two node input layer (Layer 1) takes as input the physiological parameters Lₚ and K. These inputs feed to the two hidden layers (Layer 2 and Layer 3) using 24 nodes and the swish activation function. The outputs of the second hidden layer (Layer 3) are then passed to the output layer (Layer 4) consisting of 21 nodes, representing the temporally discretized accumulation profile.

In table 3, the bounds on input variables, Lp and K, are used for the surrogate model construction to create ANNs in formulation (3)

TABLE 3

| | Control | | Treatment | |
|---|---|---|---|---|
| Bounds | Lower | Upper | Lower | Upper |
| Variable | bound | bound | bound | bound |
| L_p (cm/mm Hg-sec) | 1.00 × | 1.75 × | 5.00 × | 3.50 × |
| | 10\hat{ }(−7) | 10\hat{ }(−6) | 10\hat{ }(−7) | 10\hat{ }(−6) |
| K (cm [symbol] \hat{ }2 [symbol] /mm Hg-sec) | 1.00 × | 1.00 × | 7.00 × | 4.00 × |
| | 10\hat{ }(−7) | 10\hat{ }(−6) | 10\hat{ }(−7) | 10\hat{ }(−6) |

Due to the relatively small size of the ANNs, the models were trained using a combination of batch and mini-batch gradient descent with a mini-batch size of 10% of the training data set. The Adam optimizer was used for training with the standard mean-squared-error (MSE) loss function. The model was trained for 50 epochs using an early stopping criteria, with a MSE tolerance of 10⁻⁷. The learning rate was kept constant at 10⁻³. Following training, the MSE and mean relative percent error were evaluated on the test set. This training protocol was found to be effective, as indicated by the time and performance metrics listed in Table 4.

Table 4 shows the benchmark metrics for development time and performance of artificial neural network surrogate models of difference cases (70 kDa—control; 70 kDa—treatment; 500 kDa—control; 500 kDa—treatment) are reported. “70 kDa” and “500 kDa” denote molecular weights of nanocarriers. “Treatment” denotes both 3 mg/kg and 30 mg/kg dexamethasone (DEX) treatment.

TABLE 4

| | Time Metrics | | Performance Metrics | |
|---|---|---|---|---|
| | Data | | Mean- | Mean- |
| | Generation | Training | Squared | Percent |
| Case | (s) | (s) | Error | Error (%) |
| 70 kDa-- | 166 | 473 | 5.49 × 10\hat{ }(−7) | 0.339 |
| Control | | | | |
| 70 kDa-- | 166 | 538 | 2.32 × 10\hat{ }(−7) | 0.102 |
| Treatment | | | | |
| 500 kDa-- | 166 | 474 | 3.23 × 10\hat{ }(−7) | 0.467 |
| Control | | | | |
| 500 kDa-- | 166 | 539 | 1.55 × 10\hat{ }(−7) | 0.096 |
| Treatment | | | | |

In previous studies, recurrent neural network model architectures are utilized as a typical method to simulate dynamical systems by directly approximating the numerical integration function as opposed to the entire numerical integration procedure. This method was not employed herein as it would necessitate an iterative loop in the objective function (due to the feedback of information of earlier-time states) to create the concentration profile for each function evaluation. Such a process would introduce additional complexity that would negatively impact the solution times when included in deterministic global optimization routines used in this work.

Simplified Parameter Estimation Problem: A simplified parameter estimation problem is proposed using ANN surrogate models introduced in the above disclosure directed to Machine Learning Model. Similar to (1), the SSE is minimized between the average concentration predicted by the ANN surrogate model and experimental data over the entire time horizon, with inequality constraints on superficial IFP:

$${min}_{π ∈ \prod } {\sum }_{i = 1}^{n} {( {\hat{c}}_{avg , i}^{ANN} ( π ) - {\hat{c}}_{avg}^{data} ( {t}_{i} ) )}^{2} \\ s . t . \\ {\hat{p}}_{peri} ( π ) \leq {\hat{p}}_{peri , ma x} \\ {\hat{p}}_{peri} ( π ) \geq {\hat{p}}_{peri , m i n} , \qquad ( 3 )$$

where ĉ^{ANN} represents the dimensionless spatial average nanocarrier concentration at discrete time node i calculated from the ANN model. The inequality constraints on the superficial IFP may be simplified and reformulated as equivalent inequalities that are linear in the optimization variables (model parameters) Lₚ and K utilizing the closed-form analytical solution for the IFP profile from the prior art. This simplifies the problem significantly and, in turn, reduces the computational complexity of solving (3). The details of how this is done can be found in Supplemental Example 3.

The optimization formulation (3) can then be reformulated as:

$${min}_{π ∈ \prod } {\sum }_{i = 1}^{n} {( {\hat{c}}_{avg , i}^{ANN} ( π ) - {\hat{c}}_{avg}^{data} ( {t}_{i} ) )}^{2} \\ s . t . \\ {π}_{2} \leq {ζ}_{ma x} {π}_{1} \\ {π}_{2} \geq {ζ}_{m i n} {π}_{1} , \qquad ( 4 )$$

where ζₘₐₓ and ζₘᵢₙ are listed in Table 5 and are calculated based on the physical bounds on the superficial IFP listed in Table 1. The calculation procedure is described in Supplemental example 1.

In Table 5, the coefficients for the constraints on the superficial (peripheral) IFP are calculated for the control, 3 mg/kg, and 30 mg/kg DEX treatment cases in formulation (3) based on the IFP bounds previously reported. These values are used to ensure physically meaningful solutions are identified.

TABLE 5

| Dose | Control | 3 mg/kg | 30 mg/kg |
|---|---|---|---|
| ζ_ [symbol] min [symbol] (cm) | 0.2855 | 0.7355 | 1.5898 |
| ζ_ [symbol] max [symbol] (cm) | 0.3967 | 1.0577 | 2.4447 |

TME-Normalizing Therapy Design for Dose Selection: A method is disclosed for optimal TME-normalizing therapy design for dose selection with the overall objective of improving transport and accumulation of anticancer drugs within the tumor interstitium. To do so, the experimental effects are investigated of different doses of pretreatment DEX and utilize empirical correlations for optimal decision-making. Empirical correlations are required to construct a mathematical relationship between DEX dose and two important physiological parameters: vascular hydraulic conductivity Lₚ and interstitial hydraulic conductivity K. A systematical mathematical methodology for TME-normalizing therapy design is disclosed.

Based on the obtained preclinical data, nonlinear regression is utilized with a rational model to establish the following relationships:

$${f}_{{L}_{p}}^{r} ( x ) = \frac{- 7.5 \; 19 \times {10}^{- 8} {x}^{2} + 3.355 \times {10}^{- 6} x + 6.944 \times {10}^{- 7}}{x + 0.6175} , \qquad ( 5 )$$

$${f}_{K}^{r} ( x ) = \frac{- 2.458 \times {10}^{- 8} {x}^{2} + 2.524 \times {10}^{- 6} x + 2.916 \times {10}^{- 7}}{x + 0.7816} , \qquad ( 6 )$$

where x denotes pretreatment DEX dose

$$( {mg}_{∠} / kg ) ,$$

and the functions f^{r} and f^{r} represent the values of Lₚ and K, respectively, following treatment with DEX, as predicted by the rational regression model.

Since the obtained data are limited to the three pretreatment DEX doses, different dose-dependent relationships could exist with other data sets. This demonstrates the applicability of the disclosed method with fictitious experimental data exhibiting complicated dose-dependent treatment relationships for pretreatment DEX doses between the actual data of 3 mg/kg and 30 mg/kg with dosages set at 10 mg/kg, 15 mg/kg, 20 mg/kg, and 25 mg/kg.

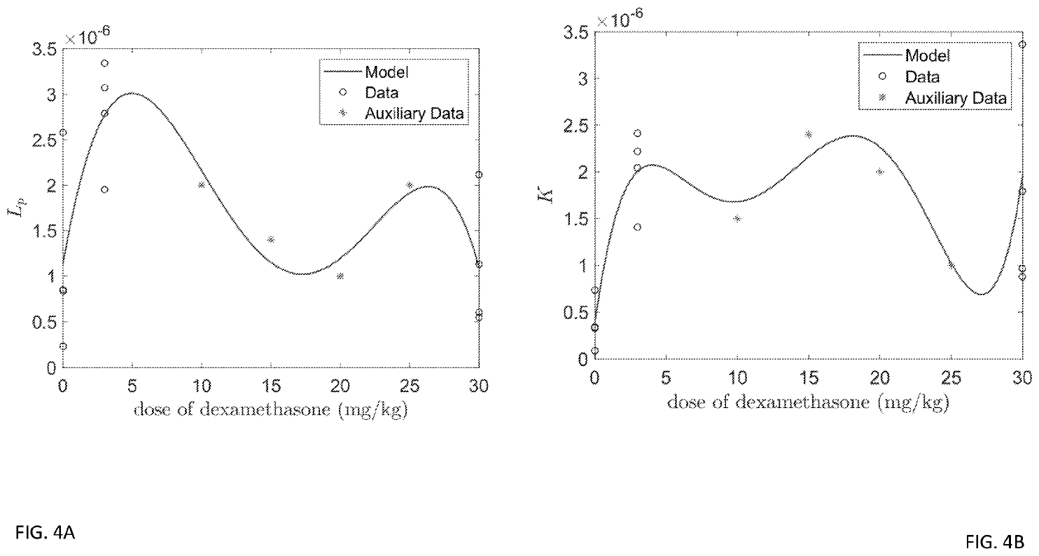

The original data, fictitious data, and corresponding polynomial regression models are plotted in FIG. 4A and B. The regression equations are given by:

$${f}_{{L}_{p}}^{p} ( x ) = - 6.23 \times {10}^{- 13} {x}^{5} - 5.96 \times {10}^{- 11} {x}^{4} + 5.61 \times {10}^{- 9} {x}^{3} - 1.272 \times {10}^{- 7} {x}^{2} + 8.797 \times 1 {0}^{- 7} x + 1.131 \times {10}^{- 8} , \qquad ( 7 )$$

$${f}_{K}^{p} ( x ) = 1.139 \times {10}^{- 11} {x}^{5} - 8.389 \times {10}^{- 10} {x}^{4} + 2.183 \times {10}^{- 8} {x}^{3} - 2.421 \times {10}^{- 7} {x}^{2} + 1.093 \times {10}^{- 8} x + 3.798 \times {10}^{- 7} , \qquad ( 8 )$$

where x is the DEX dose as before, and f^{j}_{Lp} and f^{j}_{K} represent the values of Lₚ and K, respectively, following treatment with DEX, as predicted by the polynomial regression model.

FIGS. 4A and 4B show experimental data and corresponding regression models for (7) and (8) are respectively plotted in FIG. 4A Lₚ versus dose; In FIG. 4B K versus dose of dexamethasone. Auxiliary, fictitious data are considered to demonstrate the applicability of the proposed approach to complex dose-dependent relationships that could exist naturally.

The TME-normalizing therapy design problem is formulated as the following NLP:

$${max}_{x ∈ X} {\hat{c}}_{avg} ( {t}_{f} , ( {f}_{{L}_{p}}^{j} ( x ) , {f}_{K}^{j} ( x ) ) , {d}_{m} ) . \qquad ( 9 )$$

with j∈{r, p}. The objective is to seek an optimal dose that maximizes the spatial average nanocarrier concentration ĉ_{avg} in the tumor interstitium at t_{f}=5 min. The function ĉ_{avg} is evaluated by the numerical solution of the solute transport model, and the correlations between hydraulic conductivities and DEX doses are established as (5) and (6) for the existing data, and (7) and (8) for the fictitious data.

Drug Size Design: Practicability of the tumor transport model for drug size design problems is addressed. After the optimal dose of pretreatment DEX is determined and a patient's response to that treatment is quantified, an anticancer nanocarrier is designed that results in optimal delivery to the tumor interstitium. For example, a nanoparticle size can be tuned for a patient-specific tumor pathophysiology.

After DEX pretreatment, it is desirable to determine an optimal nanocarrier size that can maximize the drug concentration in the interstitial space of the tumor. Alteration in pharmacokinetics, such as distribution and excretion, can have a substantial influence on achieving the desired therapeutic concentration of a particular nanocarrier. A very high concentration may result in side effects or toxicity. A very low concentration will be ineffective. In this situation, an optimal therapy requires a strict guarantee of some safety/performance specifications. The drug size design problem is formulated as a PDE-constrained NLP to account for these potential requirements:

$${max}_{{d}_{m} ∈ Z} {\hat{c}}_{avg} ( {t}_{f} , π , {d}_{m} ) \\ s . t . \\ {\hat{c}}_{peri} ( {t}_{f} , π , {d}_{m} ) \leq {λ}_{1} \\ {\hat{c}}_{peri} ( {t}_{f} , π , {d}_{m} ) \geq {λ}_{2} . \qquad ( 10 )$$

where t_{f} is the final time (t_{f}=5 min), ĉₚₑᵣᵢ is the dimensionless nanocarrier concentration in the superficial area of tumor (g/mL). The mappings between physiological parameters that are directly related to the nanocarrier sizes are established and introduced in the disclosure below, directed to Relationship Between Nanocarrier Size And Physiological Parameters. For this disclosure, the relationship λ₁=4.5 is utilized as the threshold for the safety constraint, which is double the periphery nanocarrier concentration for the 3 mg/kg DEX treatment case. Further, the relationship λ₂=3.6 is utilized as the performance constraint, which is chosen based on periphery nanocarrier concentration for the control case. These thresholds are merely chosen for demonstrating the drug size design approach and how to deal with the situation that a design is implemented under potential performance/safety requirements.

Furthermore, a therapy design strategy is proposed that simultaneously seeks an optimal dose of DEX and an optimal nanocarrier size that maximizes the nanocarrier concentration accumulation inside the tumor interstitial space:

$${max}_{x ∈ X , {d}_{m} ∈ Z} {\hat{c}}_{avg} ( {t}_{f} , ( {f}_{{L}_{p}}^{j} ( x ) , {f}_{K}^{j} ( x ) ) , {d}_{m} ) \\ s . t . \\ {\hat{c}}_{peri} ( {t}_{f} , ( {f}_{{L}_{p}}^{j} ( x ) , {f}_{K}^{j} ( x ) ) , {d}_{m} ) \leq {λ}_{1} \\ {\hat{c}}_{peri} ( {t}_{f} , ( {f}_{{L}_{p}}^{j} ( x ) , {f}_{K}^{j} ( x ) ) , {d}_{m} ) \geq {λ}_{2} . \qquad ( 11 )$$

with j∈{r, p}. This formulation provides an alternative methodology for neoadjuvant therapy that could identify a possible therapy and nanocarrier size combination that leads to improved transport and accumulation over the individual results determined by the sequential design approach.

ANN surrogate models are also proposed for the simultaneous design problem (11) to reduce the computational burden over the PDE-constrained problem. To accomplish this, two ANNs are established each with Lₚ, K, and dₘ as inputs. The respective ANNs each have a single output ĉ^{ANN}_{avg} and ĉ^{ANN}ₚₑᵣᵢ. These ANN models are different from the one in the above disclosure directed to Machine Learning Model. To train these new ANNs, a Sobol sequence sampling method was used again to create a 10⁶ point data set on the domain (Lp, K, dₘ)∈[5×10⁻⁷, 5×10⁻⁶]×[5×10⁻⁷, 5×10⁻⁶]×[10, 60]. Consistent with the parameter estimation of the ANN models, the data set was scaled using a min-max normalization and divided randomly into training (70%) and validation (30%) sets. Training was performed using Flux.jl with the Adam optimizer with a learning rate of 10⁻⁴. Each ANN model for ĉ^{ANN}_{avg} and ĉ^{ANN}ₚₑᵣᵢ has two hidden layers with 12 neurons using the tanh activation function. The models were trained using an equivalent protocol to that described in above in the disclosure directed to Machine Learning Model. The benchmarks for data generation, training time, and performance are shown in Table 6.

In Table 6, the table provides benchmark metrics of time and performance (data generation time, training time, mean-squared error and mean-percent error) for ANN surrogate model development in (12) (below).

TABLE 6

| | Time Metrics | | Performance Metrics | |
|---|---|---|---|---|
| | Data | | Mean- | Mean- |
| | Generation | Training | Squared | Percent |
| Case | (s) | (s) | Error | Error (%) |
| 70 kDa-- | 166 | 473 | 5.49 × 10\hat{ }(−7) | 0.339 |
| Control | | | | |
| 70 kDa-- | 166 | 538 | 2.32 × 10\hat{ }(−7) | 0.102 |
| Treatment | | | | |
| 500 kDa-- | 166 | 474 | 3.23 × 10\hat{ }(−7) | 0.467 |
| Control | | | | |
| 500 kDa-- | 166 | 539 | 1.55 × 10\hat{ }(−7) | 0.096 |
| Treatment | | | | |

The formulation with ANN models for the simultaneous design approach can be expressed as:

$${max}_{x ∈ X , {d}_{m} ∈ Z} {\hat{c}}_{avg}^{ANN} ( ( {f}_{{L}_{p}}^{j} ( x ) , {f}_{K}^{j} ( x ) ) , {d}_{m} ) \\ s . t . \\ {\hat{c}}_{peri}^{ANN} ( ( {f}_{{L}_{p}}^{j} ( x ) , {f}_{K}^{j} ( x ) ) , {d}_{m} ) \leq {λ}_{1} \\ {\hat{c}}_{peri}^{ANN} ( ( {f}_{{L}_{p}}^{j} ( x ) , {f}_{K}^{j} ( x ) ) , {d}_{m} ) \geq {λ}_{2} . \qquad ( 12 )$$

Settings for Solving Optimization Problems: The settings used for the numerical methods and software packages are discussed herein. For the parameter estimation, TME-normalizing therapy design, and drug size design problems, the spatial domain for both fluid transport and solute transport models are discretized into N=100 nodes. The simulation time horizon contains 21 time nodes (5 min). Based on the superficial region (around 0.07 mm from surface) and the average tumor diameter (0.6-1.1 cm) in the DEX treatment research, the relationship n=99 is selected to account for the superficial region of the tumor. The physiological parameters used in the tumor transport model are provided in Table 7. The parameter estimation, drug size design, and TME-normalizing therapy design problems are all solved to global optimality using the EAGO v0.6.1 solver via JUMP v0.21.4 in the Julia programming language. Custom bounding routines with the mixed IA/AA method and standard DI are utilized in the BnB algorithm for solving the parameter estimation and drug size design problems. For the parameter estimation problems, the absolute global convergence tolerance is set as 10⁻⁶, and the relative global convergence tolerance is set as 10⁻¹ for each case. For the drug size design and TME-normalizing therapy design problems, the absolute convergence tolerance is set as 10⁻⁶, and the relative convergence tolerance set as 10⁻². Each problem was run on a personal workstation with an Intel Xeon E3-1270v5 4-core/8-thread CPU operating at 3.60 GHz/4.00 GHz (base/turbo) frequency and 32 GB ECC RAM running Windows 10 Version 2004.

In Table 7, physiological parameter values are reported for constructing the tumor transport model discussed below in the portion of the disclosure directed to Tumor Transport Model,. “70 kDa” and “500 kDa” denote molecular weights of nanocarriers.

TABLE 7

| Parameter | Definition | Value |
|---|---|---|
| S/V (cm⁻¹) | Vascular density | 200 |
| D (cm²/sec) | Diffusion coefficient | 2 × 10\hat{ }(−7) (70 kDa); |
| | | 1.375 × 10\hat{ }(−7) (500 kDa) |
| p_v (mm Hg) | Vascular pressure | 25 |
| k_d (min) | Blood circulation time | 1480 (70 kDa); 1278 (500 kDa) |
| μ (mm Hg-sec) | Blood viscosity | 3 × 10\hat{ }(−5) |
| L (μm) | Vessel wall thickness | 5 |
| γ | Fraction of pore area | 1 × 10\hat{ }(−3) |

### Example 1: Global Optimization Results for Model Validation

The global optimization results are discussed for the parameter estimation problems. The global optimal solutions obtained from the parameter estimation problems for different doses of DEX treatment cases are listed in Table 8 for each formulation with the original mechanistic tumor transport model (1) as well as the ANN surrogate model (4). Dose selection for the experiments was based on previous work, which confirmed 3 mg/kg DEX as the lowest dose to reduce IFP. Additionally, this dose is similar to that used in the clinical trials of CDDP/m (NCT02043288). The global solutions found for both the mechanistic model and ANN model are very close to one another, with the relative error for each case being within 2.5%. This certifies the accuracy of the ANN surrogate models and the validity of the inequality constraints simplifications. Previously, local optima are obtained for the parameter estimation problems. In that work, it was found that the estimated Lp value for 3 mg/kg DEX treatment case with 500 kDa nanocarrier injection exhibited a decreasing trend from the control case, whereas an increasing trend is obtained. This does not represent a contradiction, as the parameter estimation problems differ significantly in that they consider different simulation time horizons. Furthermore, in the case of the prior art, no inequality constraints on the IFP were considered.

Table 8 shows global optima for parameter estimation problems using the mechanistic model (1) and the ANN model (4). Solutions obtained for the ANN surrogate model are very close to those obtained for the mechanistic model. This is to be expected since a high-degree of accuracy of the ANN was obtained when training. The unit for L*p is cm/mm Hg-sec and for K* is cm²/mm Hg-sec.

TABLE 8

| | Dextran molecular weight | | | | | |
|---|---|---|---|---|---|---|
| | 70 kDa | | | 500 kDa | | |
| Dose | Control | 3 mg/kg | 30 mg/kg | Control | 3 mg/kg | 30 mg/kg |
| P_eff (cm/sec) | 9.60 × 10\hat{ }(−7) | 4.61 × 10\hat{ }(−6) | 2.80 × 10\hat{ }(−6) | 8.18 × 10\hat{ }(−7) | 4.30 × 10\hat{ }(−6) | 1.62 × 10\hat{ }(−6) |
| L_p\hat{ } * - | 8.51 × 10\hat{ }(−7) | 2.80 × 10\hat{ }(−6) | 1.12 × 10\hat{ }(−6) | 8.62 × 10\hat{ }(−7) | 2.22 × 10\hat{ }(−6) | 7.50 × 10\hat{ }(−7) |
| mechanistic | | | | | | |
| model | | | | | | |
| L_p\hat{ } * - | 8.39 × 10\hat{ }(−7) | 2.77 × 10\hat{ }(−6) | 1.12 × 10\hat{ }(−6) | 8.43 × 10\hat{ }(−7) | 2.22 × 10\hat{ }(−6) | 7.50 × 10\hat{ }(−7) |
| ANN model | | | | | | |
| K\hat{ } * - | 3.35 × 10\hat{ }(−7) | 2.03 × 10\hat{ }(−6) | 1.80 × 10\hat{ }(−6) | 3.34 × 10\hat{ }(−7) | 2.34 × 10\hat{ }(−6) | 1.21 × 10\hat{ }(−6) |
| mechanistic | | | | | | |
| model | | | | | | |
| K\hat{ } * - | 3.32 × 10\hat{ }(−7) | 2.04 × 10\hat{ }(−6) | 1.78 × 10\hat{ }(−6) | 3.29 × 10\hat{ }(−7) | 2.36 × 10\hat{ }(−6) | 1.20 × 10\hat{ }(−6) |
| ANN model | | | | | | |

The time costs for each model are reported in Table 9. For DEX treatment cases, the parameter estimation problems with the mechanistic model and the proposed customized bounding routines are extremely computationally expensive. Despite using the most efficient global bounding method considered, these problems still required hours or even days to solve. In contrast, the parameter estimation problems for DEX treatment cases using the ANN surrogate models can be solved within one minute. Even accounting for the time costs of generating data and training, the ANN surrogate models significantly reduce the burden of solving the parameter estimation problems to global optimality. Interestingly, it takes about an order-of-magnitude longer to solve the parameter estimation problems for the control cases with ANN models versus the mechanistic models. In these cases, it is observed that the lower-bounding problems solved for the ANN problems furnish weaker bounds than for the mechanistic modeling case, resulting in slower convergence of the BnB algorithm.

Table 9 shows computational time costs for the parameter estimation problems using the mechanistic model (1) and the ANN model (4). Barring the control case, solving the PDE-constrained optimization problem (1) requires significantly more time than the problem with the ANN (4), which does not account for the ANN training time.

TABLE 9

| | Dextran molecular weight | | | | | |
|---|---|---|---|---|---|---|
| | 70 kDa | | | 500 kDa | | |
| Dose | Control | 3 mg/kg | 30 mg/kg | Control | 3 mg/kg | 30 mg/kg |
| Mechanistic model (s) | 8.5 | 169558.3 | 238732.2 | 8.1 | 398792.2 | 50368.1 |
| ANN model (s) | 97.9 | 7.3 | 25.8 | 23.2 | 17.6 | 18.8 |

### Example 2: Interstitial Fluid Pressure and Velocity Profiles

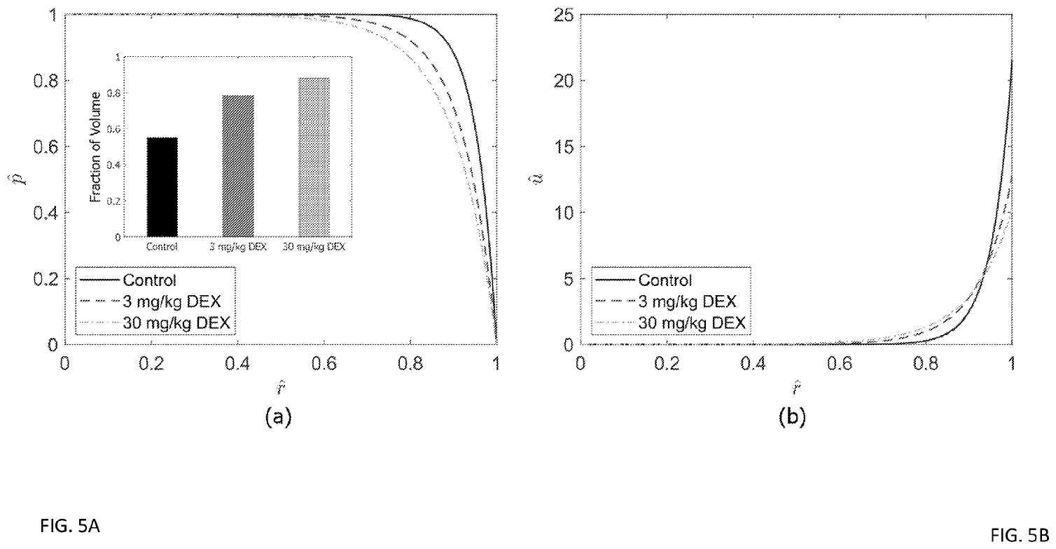

Previous studies showed that an important barrier to drug delivery in the TME is the elevated IFP resulting in reduced pressure gradients across the vessel wall. This is due to the interstitial hypertension phenomenon caused by leaky blood vessels and the lack of functional lymphatic vessels, which drain excess fluid from tumor tissue. TME-normalizing therapy can repair the abnormal vasculature and reduce IFP, resulting in a higher pressure gradient for higher transvascular and interstitial fluid flow. Thus, the IFP i29uantifyied with different doses of DEX treatment to characterize the TME normalization process. The dimensionless IFP profiles as functions of dimensionless radial position with respect the optimal solutions (i.e., from Table 8) are illustrated in FIG. 5A. The IFP profiles tend to reach a steady-state pressure pₛₛ at the center of the tumor. where the IFP equals the vascular pressure pᵥ. However, in the periphery, the IFP rapidly decreases with increasing distance from the tumor center. This finding is consistent with previous mathematical models and experimental findings. Thus, the IFP profiles indicate that the extravasation of fluid from blood vessels is extremely slow near the center, whereas it is highest at the periphery due to lower IFP leading to an increased transvascular pressure gradient. In addition, the model confirms that DEX reduces the spatially-averaged IFP and therefore establishes a more advantageous transvascular pressure gradient that contributes to enhanced transvascular fluid flow, that will further affect the interstitial fluid transport.

FIGS. 5A and 5B show radial dose-dependent interstitial fluid pressure and velocity profiles. FIG. 5A shows mathematical model-generated profiles of dimensionless interstitial fluid pressure (IFP) p [symbol] =(p−p_{∞})/(pₛₛ−p_{∞}) versus the dimensionless tumor radial position r [symbol] from vessel permeability data collected using fluorescently-labelled 500 kDa dextran in control tumors and tumors in mice treated with 3 mg/kg and 30 mg/kg dexamethasone (DEX) daily for four days are presented. Spatially-averaged IFP is reduced with DEX treatment. The interior bar graph illustrates the fraction of tumor volume that has a favorable transvascular pressure gradient (i.e. p [symbol] ≤0.9933). This IFP threshold is determined by the region with r [symbol] ≥0.6 for the 3 mg/kg DEX treatment case, which is taken as the volume with favorable transvascular pressure gradient. FIG. 5B shows normalized interstitial fluid velocities (IFV) u [symbol] =uR/(K(pₛₛ−p_{∞})) are plotted versus dimensionless tumor radial position r [symbol] . Greater IFVs are achieved deeper in the tumor interstitium following DEX treatment with a reduction in velocity nearest the tumor periphery. This results in increased interstitial transport of nanocarriers.

The interstitial fluid velocity (IFV) is generated from the interior IFP gradient by Darcy's law (introduced in Supplemental Example 1.1). To investigate the effects of TME normalization on interstitial fluid transport, the normalized IFV (u [symbol] =uR/(K(pₛₛ−p_{∞}))) profiles are quantified for different doses of DEX. A positive value of IFV indicates that the interstitial fluid flow is from the center to the periphery of the tumor. As illustrated in FIG. 5(b), the normalized IFV is very low around the center and increases towards the periphery where there is the highest flow rate. The dimensionless parameter α=R√(L_p S\/KV) (introduced in Supplemental Example 1.1), which is a measure of the ratio of interstitial to vascular resistances of fluid flow, represents the gradient of increase of normalized IFV. Summarily, a larger value for α indicates a steeper increase in the normalized IFV profile with increasing distance from tumor center. The model-predicted α values for the control. 3 mg/kg DEX treatment, and 30 mg/kg DEX treatment cases from the 500 kDa dextran experimental data are 22.521. 13.756 and 11.219, respectively. Thus, compared to the control, the treated cases have smaller values of α that indicate a gradual increase in normalized IFV from the center tumor over a larger fraction of tumor volume. Normalized IFV neglects the influence of interstitial hydraulic conductivity K. However, K is larger by an order of magnitude for DEX treated cases than the control case (Table 8). Thus, the actual IFV for DEX treated cases is always higher than the control case. Although, DEX treatment increases the perfused vascular density, the tumor radius R and vascular density S/V do not vary significantly between each case. Thus, a reduction in the ratio of the vascular hydraulic conductivity to the interstitial hydraulic conductivity (i.e., Lₚ/K) is the major reason for a reduction in α. A smaller value of Lₚ/K indicates a larger proportion of interstitial fluid transport. Therefore, a less steep normalized IFV profile resulting from a smaller α caused by a reduction of Lₚ/K implies enhanced interstitial fluid transport by vascular and ECM normalization.

### Example 3: Solute Concentration Profiles

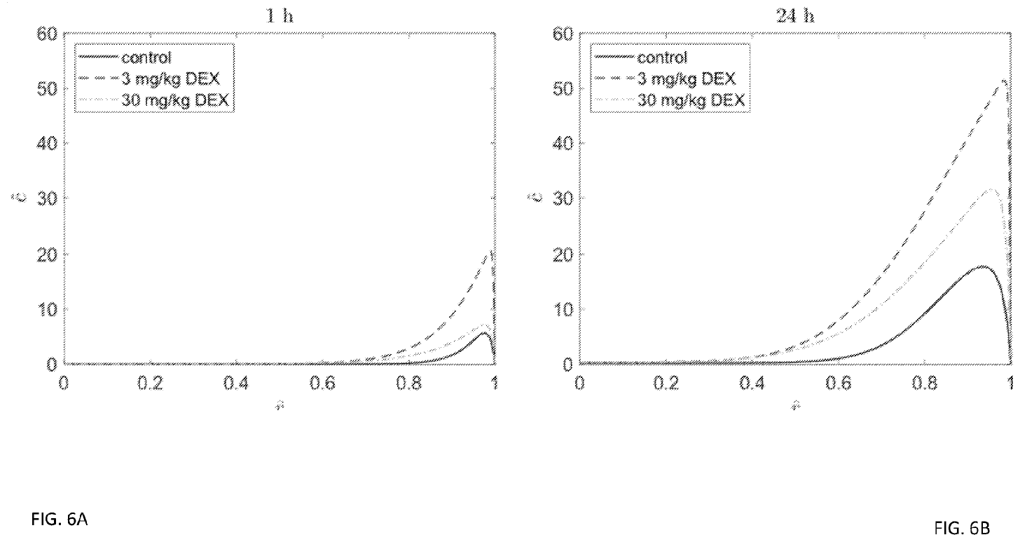

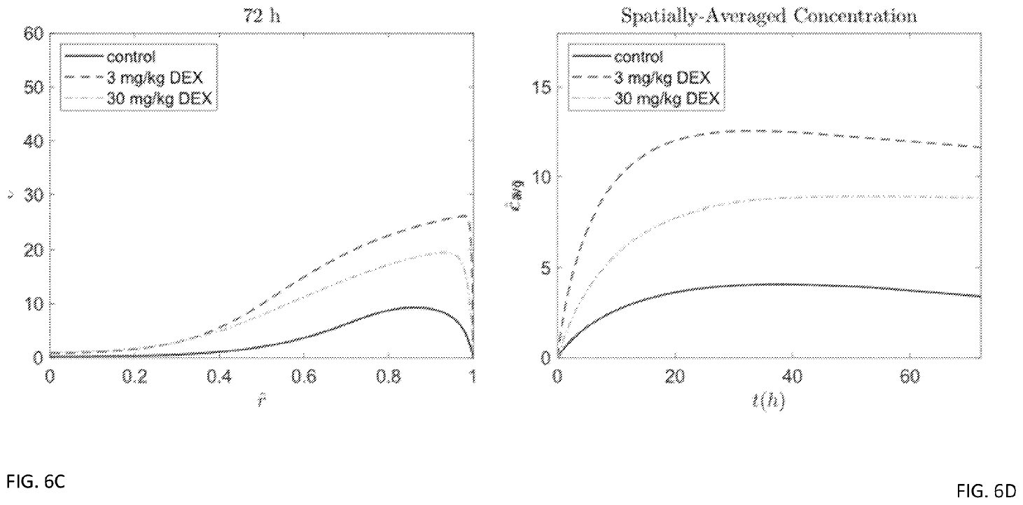

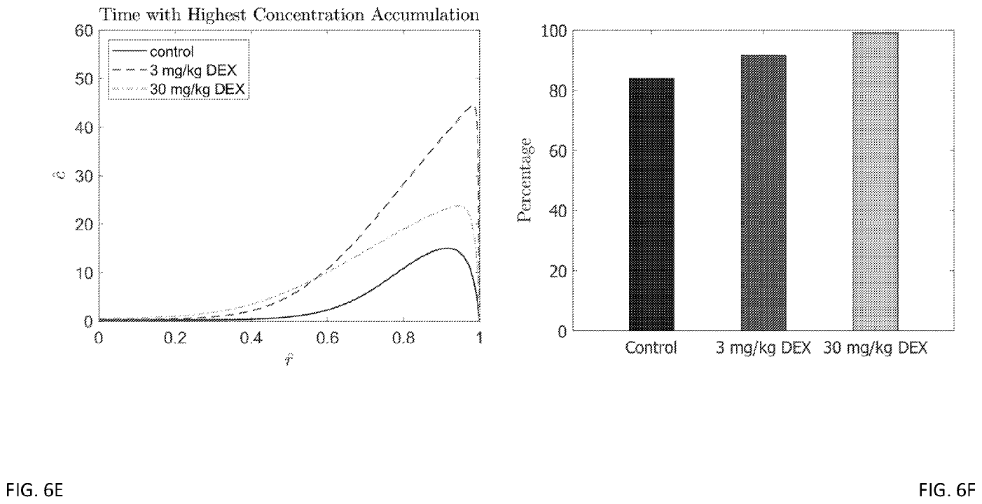

Drug distribution is determined within tumors by obtaining solute concentration profiles from the IFP and IFV profiles. The IFP gradient induces transvascular convective transport, the IFV profiles reflect interstitial convective transport, and the solute concentration gradient induces interstitial diffusive transport. FIG. 6 illustrates the model-predicted solute concentration profiles with respect to dimensionless tumor radial position r [symbol] for the 500 kDa dextran experimental data. with the vascular concentration following an exponential decay post-administration.

FIGS. 6A-6D show radial and temporal dose-dependent solute concentration profiles. Interstitial concentrations ĉ of 500 kDa dextran are plotted versus dimensionless tumor radial position r [symbol] for a vascular concentration with a half-life of around 21 h for the control, 3 mg/kg DEX treatment, and 30 mg/kg DEX treatment cases at 1 h in FIG. 6A; at 24 h in FIG. 6B; and at 72 h post-administration in FIG. 6C. FIG. 6D shows spatially-averaged interstitial concentrations ĉ_{avg} are plotted versus time. After DEX treatment, the overall solute concentration accumulation is increased inside the tumor. The 3 mg/kg DEX treatment case results in the highest overall concentration accumulation.

As illustrated in FIG. 6A, the interstitial concentration at 1 h post-administration of the dextran is equal to the normal tissue concentration (equals 0 in dimensionless form) at the periphery and quickly increases to a peak in the peripheral region where there is a higher transvascular pressure gradient, which significantly enhances transcapillary convective solute transfer. The fraction of tumor volume that has a higher transvascular pressure gradient is graphed for each treatment group in the inset of FIG. 5A. Simultaneously, the higher IFV in the peripheral region causes a higher interstitial fluid flux that carries the solute outwards to the periphery. As a result. the solutes accumulate and reach peak concentration near the periphery. then decrease to zero at around r [symbol] =0.8 for the control case and r [symbol] =0.6 for the 3 mg/kg and 30 mg/kg DEX treatment cases. Indeed, the region with favorable transvascular pressure gradient for DEX treated cases is larger than the control case (FIG. 5A). This pressure gradient leads to an enhanced convective transvascular transport that carries solutes into the interstitial space of a larger proportion of the tumor. In other words, the region with higher solute accumulation occurs over a longer fraction of tumor radius for the DEX treatment cases compared to control.

As presented in FIG. 6A, the interstitial concentration profiles for all treatment cases have higher peaks at 24 h than 1 h. The concentration peaks for all cases at 72 h (FIG. 6C) are lower than 24 h but higher than 1 h. This is because the vascular concentration decays at 72 h compared with 24 h so that there are fewer nanocarriers to be carried by transvascular flow into the interstitial space. In addition, the interstitial concentration profiles at 72 h become flatter than 24 h with a higher concentration retained towards the middle of the tumors, such as at r [symbol] =0.5. This is caused by the slower interstitial diffusion generated from the concentration gradient that gradually transfers nanocarriers from the concentration peak in the periphery towards the tumor center, where the concentration of nanocarriers is near zero. The transvascular flow is limited at 72 h due to the systemic clearance of circulating nanocarriers, but the diffusion caused by concentration gradient becomes more evident in the flatter concentration profiles.

As illustrated in FIG. 6D, the spatially-averaged interstitial concentration rises to a peak and stays steady after that. Although the vascular concentration of nanocarriers decays exponentially. the spatially-averaged interstitial concentrations decrease slowly after reaching the peak. The concentration profiles at the time with respect to the highest spatially-averaged concentration accumulation are illustrated in FIG. 6E, discussed below. Highest spatially-averaged concentration occurs at 38.8 h. 34.2 h and 53.9 h for the control, 3 mg/kg and 30 mg/kg DEX treatment cases, respectively. In general, the nanocarriers accumulate to a peak concentration in the first dozens of hours and then decrease with a slow rate.

FIG. 6E shows the radial interstitial concentration profiles at the time corresponding to the highest spatially-averaged concentrations with respect to control (38.8 h), 3 mg/kg dexamethasone treatment (34.2 h), and 30 mg/kg DEX treatment (53.9 h) cases are presented.

The spatially-averaged concentrations at 72 h are 84%, 92% and 99% of their highest concentrations for the control, 3 mg/kg and 30 mg/kg DEX treatment cases, respectively (illustrated in FIG. 6f, discussed below). Spatially-averaged concentrations of the 500 kDa dextran in control tumors only decrease by 16% in 33.2 h after reaching highest concentration, indicating a retention effect. The 3 mg/kg and 30 mg/kg DEX treatments both enhance this retention effect (92% and 99% are higher than the control case). Though the 3 mg/kg DEX treatment does not result in the highest percentage of retention at 72 h (92%<99%), it has the highest spatially-averaged concentration throughout the whole time horizon. In contrast, the control case has the lowest percentage and also the lowest spatially-averaged concentration. Thus, these findings demonstrate that DEX treatment not only increases permeability but also retention towards promoting the EPR effect.

FIG. 6F shows the percentages of the spatially-averaged concentrations at 72 h over the respective highest spatially-averaged concentrations for the control, 3 mg/kg and 30 mg/kg dexamethasone (DEX) treatment cases. The DEX treatment enhances the retention effect.

The relation between the solute concentration distribution over time and dose of DEX treatment is determined. The concentration profile for the 30 mg/kg DEX treatment case is closer to the control case at 1 h post-administration, whereas it is closer to the 3 mg/kg DEX treatment case at 72 h post-administration. At 1 h post-administration, there are many nanocarriers in perfused vessels and they are carried into the tumor tissue by transvascular flow. A larger vascular hydraulic conductivity Lₚ indicates higher transvascular flow rate. However, Lₚ for 30 mg/kg DEX treatment case is closer to the control case (Table 8). Although the vessels are normalized after 30 mg/kg DEX treatment. there is too much pericyte coverage that reduces the vessel wall pore size thereby limiting nanocarrier extravasation at 1 h post-administration. In contrast, the extravasation of nanocarriers is trivial at 72 h due to the decay of its concentration in the vasculature and the interstitial concentration profile has already reached a peak and decreased. Thus, the interstitial diffusive transport becomes more prominent. Since both 3 mg/kg and 30 mg/kg DEX treatment similarly reduce hyaluronan levels and tissue stiffness, resulting in much larger interstitial hydraulic conductivity K than the control case (Table 8), the enhanced interstitial diffusive transport results in the observed profiles. In addition, the 3 mg/kg DEX treatment case results in a much higher overall nanocarrier concentration accumulation in the tumor tissue than that of the control and the 30 mg/kg cases at all time nodes (1 h. 24 h, and 72 h), indicating increased delivery of anticancer nanocarriers leading to improved efficacy as demonstrated previously. Given that the 3 mg/kg DEX treatment leads to the highest nanocarrier accumulation, the convective and diffusive transvascular fluxes are determined separately to understand how DEX increased accumulation.

### Example 4: Dexamethasone Increases Convective Transvascular Flux in Tumors

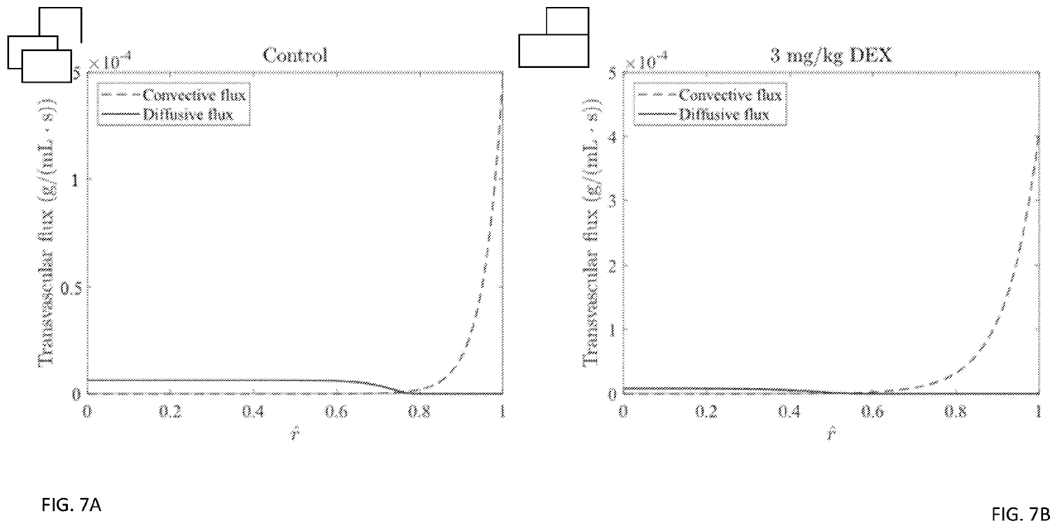

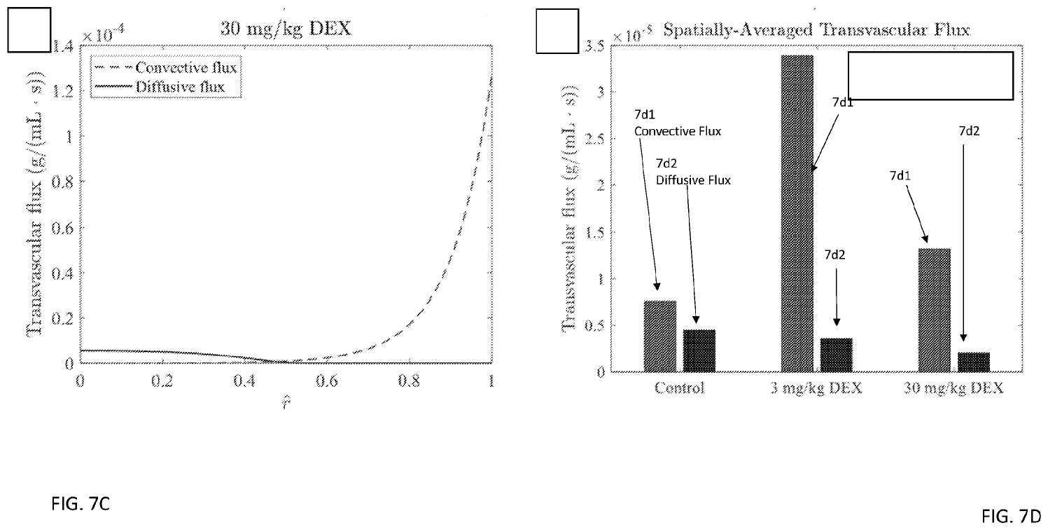

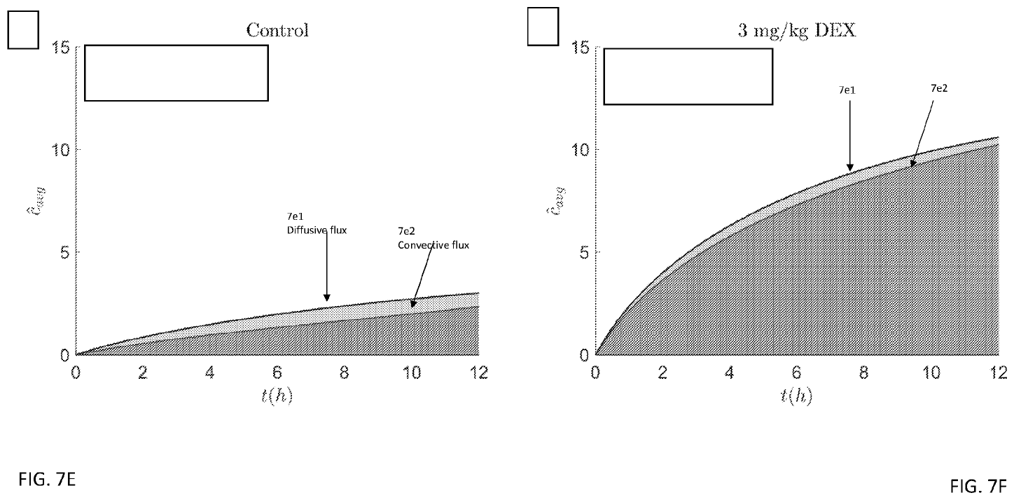

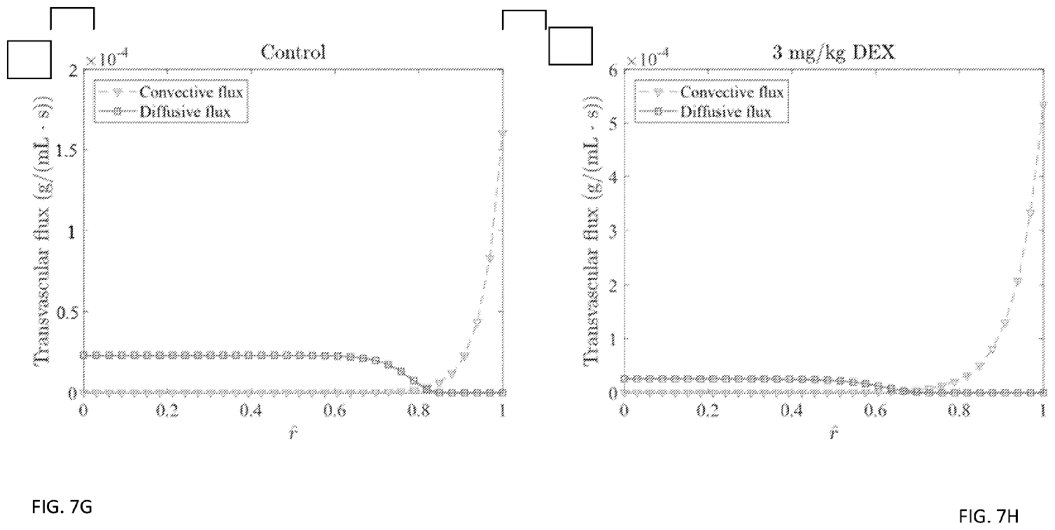

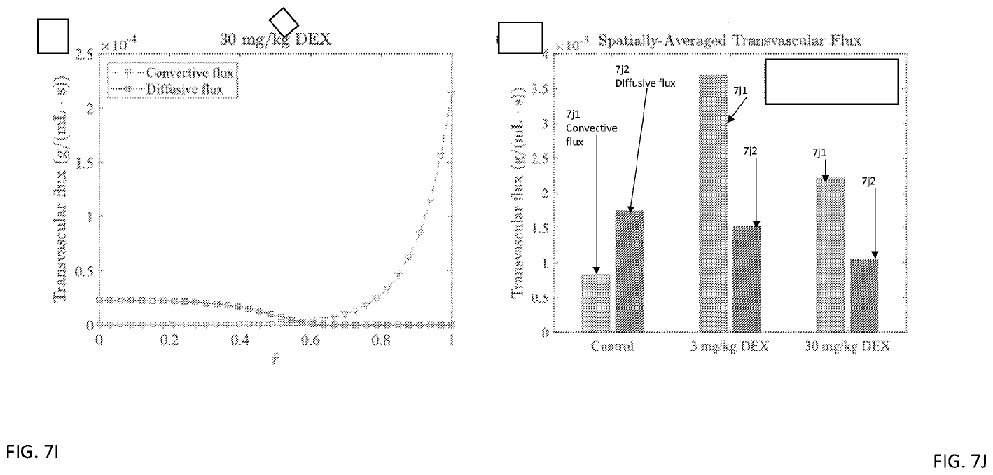

FIGS. 7A-7D show dose-dependent transvascular convective and diffusive flux profiles. The transvascular flux profiles of 500 kDa dextran over the dimensionless tumor radial position r one-hour post-administration are plotted for the control in FIG. 7a; for 3 mg/kg dexamethasone (DEX) treatment in FIG. 7b; and for 30 mg/kg DEX treatment cases in FIG. 7c. In FIG. 7d, the spatially-averaged convective flux 7d1 and diffusive flux 7d2 at one-hour post-administration for different doses of DEX are presented in this bar plot. General trends show greatest convective flux at the tumor periphery and greatest diffusive flux deeper at the tumor center. Following DEX treatment, convection accounts for a greater proportion of the spatially-averaged transvascular fluxes, demonstrating how TME normalization induces a larger transvascular pressure gradient that is advantageous for improving nanocarrier delivery in tumors. The 3 mg/kg DEX treatment induces highest convective flux, indicating that a moderate dose of DEX is more advantageous for enhancing convective transport.

After finding the interstitial concentration profiles that dictate enhanced nanocarrier distribution and accumulation with DEX treatment, the difference in concentrations between the control and DEX treatment cases depends on the relative contributions of convective and diffusive transvascular flux. Previous studies indicated that the main mechanism of transvascular transport is diffusion because elevated IFP in the TME abrogated the transvascular pressure gradient. Because DEX reduces IFP, it could enhance convective flux, which leads to more rapid transport than diffusive flux. However, the relative contributions from convection and diffusion throughout the entire volume of a tumor have never been studied before.

Based on the optimal hydraulic conductivity values determined by global optimization, the model-predicted transvascular convective and diffusive fluxes are quantified. As described in (2), the convective flux is calculated by L_p S/V (p_v−p)(1−σ)c_v and the diffusive flux is calculated by PS/V(c_v−c) Pe/(e\hat{ }Pe−1). The relative contributions from convective and diffusive flux to the spatially-averaged concentration profile with time are illustrated in FIG. 7E and 7F (discussed below). Convective flux contribution for the DEX treatment case is dominant throughout the time horizon compared with the control case. This indicates that the normalized TME after DEX treatment is advantageous for convective transport.

FIGS. 7E and 7F show the contributions from convective flux 7e1 and diffusive flux 7e2 to spatially-averaged concentrations versus time for control in FIG. 7E; FIG. 7F shows 3 mg/kg dexamethasone (DEX) treatment cases are presented. The profiles are plotted with a 12-hour horizon because the diffusive flux becomes extremely small after that. The contribution from convective flux becomes more dominant after DEX treatment.

To better understand the effects of TME normalization on transvascular transport, the spatial dependence of model-predicted diffusive and convective fluxes in tumors is determined. Previously. continuous intravital microscopy was performed on mice for one hour post-administration and investigated nanocarrier microdistribution. Here, the spatial convective and diffusive fluxes were quantified at one hour post-administration to determine their distribution in tumors. As shown in FIG. 7A, in the region with r [symbol] <0.8, the convective flux is near zero while diffusion is the main mode of transport. This is because the IFP is close to the microvascular pressure (FIG. 5A), indicating that there is no driving force for convection. Diffusion, although dominant, is small, and so there is not much transvascular flux in the tumor center. In contrast, in the region with r [symbol] >0.8, there is more convective than diffusive flux. with the latter being near zero. This convective flux at the periphery is 22-fold greater than the diffusive flux at the center. The reason for this is that the IFP in the convection-dominated region is low (FIG. 5A), which induces a high transvascular convective flux driven by a large pressure gradient. Accordingly, the convective transport increases the interstitial concentration, thereby lessening the concentration gradient and reducing the driving force for diffusion. In addition, the Pe number, which represents the ratio between transvascular convection and diffusion rates. is very large in the periphery, reflecting the extremely small diffusive flux as observed in FIG. 7A. As a result. there is a convection-dominated region and a diffusion-dominated region and the maximum rate of convective flux is an order-of-magnitude greater than the maximum diffusive flux.

Next, the effect of TME normalization on the spatial distribution of these fluxes is determined. As illustrated in FIG. 7B. the maximum convective flux at the periphery for 3 mg/kg DEX is 48-fold greater than the maximum diffusive flux, which occurs in the tumor center. Since the maximum diffusive flux is close to the control case, this indicates that convection is greatly enhanced and responsible for a larger proportion of total transvascular transport in the normalized TME after treatment with 3 mg/kg DEX. In contrast, in FIG. 7C, the maximum convective flux for 30 mg/kg DEX is 22-fold greater than the maximum diffusive flux, which is comparable to that of the control case. In fact, by comparing the values of maximum convective flux at the periphery, the flux for 30 mg/kg DEX is 14.3% less than the control case. The reason is that the vascular hydraulic conductivity Lₚ for with 30 mg/kg DEX treatment is 13% less than the control case (Table 8). This is because DEX normalizes the vessels, increases vessel maturity, and thereby reduces vessel leakiness. As a result, the vascular hydraulic conductivity reduces, leading to the lower maximum convective flux at the tumor periphery. However, these findings do not indicate that the convective flux for 30 mg/kg DEX reduces throughout the entire tumor compared to the control. This is because the volume of the convection-dominated region is much larger for DEX treatment cases.

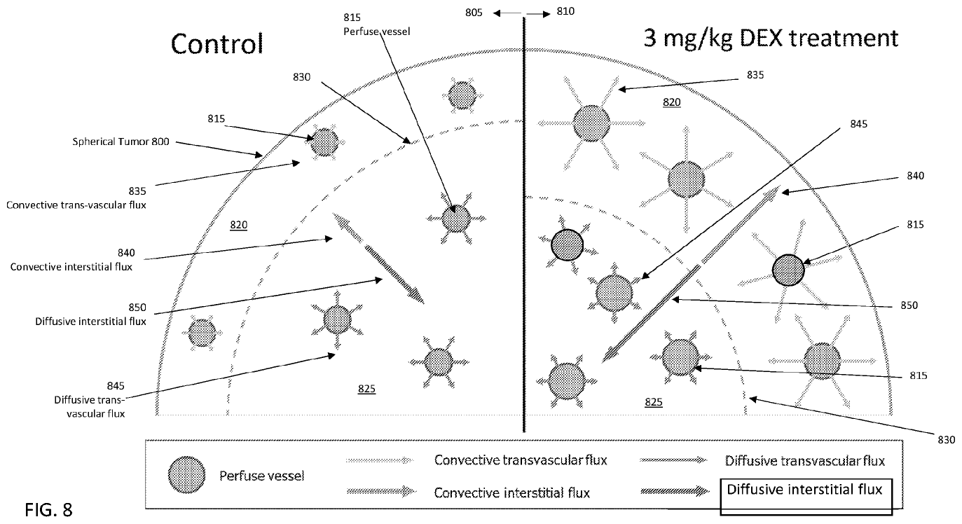

As illustrated in FIG. 7B and FIG. 7C. the convective flux for both 3 and 30 mg/kg DEX treatment cases begins to increase around r [symbol] =0.6 versus r [symbol] =0.8 for the control case, indicating a larger convection-dominated region. These findings are illustrated in FIG. 8 discussed below), which shows a schematic of tumor cross sections for the control case and 3 mg/kg DEX treatment case. 30 mg/kg DEX treatment case has almost the same proportion of convection-to-diffusion-dominated region as the 3 mg/kg case. Thus, the 3 mg/kg DEX was chosen to illustrate the treatment case in FIG. 8. The tumor volume fraction of convection-dominated region for the control case is only 49%, whereas this jumps to 78% for a tumor treated with DEX. This represents a 61% increase in the volume fraction of the tumor that is dominated by convective transport as a result of TME normalization with DEX treatment. Transvascular fluxes in FIG. 8 are scaled according to the 70 kDa dextran, and the corresponding convective and diffusive flux profiles are presented in FIGS. 7G-7J, discussed below. The findings using the 70 kDa dextran to determine the convection- and diffusion-dominated regions are consistent with those using the 500 kDa dextran. Both doses almost equally increase the volume of the convection-dominated region, but the 3 mg/kg DEX is superior because it also significantly increases the maximum convective flux.

FIGS. 7G-7J show the transvascular flux profiles over the dimensionless radius r [symbol] for the control in FIG. 7g; 3 mg/kg dexamethasone (DEX) treatment in FIG. 7H; and 30 mg/kg DEX treatment cases with 70 kDa dextran one-hour post-administration are presented in FIG. 7I. FIG. 7J shows the spatially-averaged convective flux 7j1 and diffusive flux 7j2 at one-hour post-administration for different doses of DEX are presented in this bar plot.

To quantify the contributions of convection and diffusion throughout the tumor, the spatially-averaged transvascular convective and diffusive fluxes are determined and presented in a bar plot as illustrated in FIG. 7D. A 360% increase in convective flux with 3 mg/kg DEX and a 80% increase with 30 mg/kg DEX compared to the control case. This indicates that DEX dose has a significant impact on convective transport. It turns out that a moderate dose of DEX greatly enhances convection. Excess DEX still enhances convective transport. but much less effectively. The reason is that the vascular hydraulic conductivity Lₚ for 30 mg/kg DEX is much less than 3 mg/kg DEX (Table 7). In addition, a higher dose of DEX treatment leads to a lower spatially-averaged diffusive flux (20% decrease with 3 mg/kg DEX and 65% decrease with 30 mg/kg DEX compared to control). One reason is that the elevated convective flux with DEX treatment results in a much higher interstitial concentration. Thus, the driving force from transvascular concentration gradient decreases, leading to a lower diffusive flux. In addition, that the vessel wall pore size is smaller with 30 mg/kg DEX treatment because vascular normalization reduces vessel leakiness by shrinking vessel wall pores. Accordingly, the diffusive hindrance (introduced in Supplemental Example 1.3) is also smaller. A smaller diffusive hindrance represents higher impairment to diffusion. Thus, 30 mg/kg DEX treatment results in a lower diffusive flux. In conclusion, these results demonstrate that DEX increases the accumulation of nanocarriers in tumors by increasing the convective transvascular flux. but the dose of TME normalization treatment should be titrated to avoid reducing vessel wall pore sizes that limit the benefit to enhanced convection.

FIG. 8 shows cross sections of the spherical tumor 800 are illustrated in this schematic for the control case 805 (left) and 3 mg/kg DEX treatment case 810 (right). Perfuse vessels 815 are more abundant and have a larger average diameter following DEX treatment versus the control case; a result of normalizing the tumor microenvironment. The outer shaded sections (or outer regions) 820 represent the convection-dominated region with significant pressure gradients resulting in predominant convective transvascular flux (arrows 835). The inner sections (or inner regions) 825 represent the diffusion-dominated region with almost no pressure gradient (highest interstitial fluid pressure (IFP)) resulting in predominant diffusive transvascular flux (arrows 845). The inner region 825 is much larger for the control case with the demarcation 830 (dashed curves) between regions occurring at r [symbol] =0.8, whereas the demarcation 830 between regions for the DEX treatment case is at r [symbol] =0.6. Arrows illustrate convective transvascular flux 835 (arrows directed outwardly from the perfuse vessels in the outer regions), convective interstitial flux 840 (single large arrow in each case, spaced apart from the perfuse vessels, directed radially outwardly), diffusive transvascular flux 845 (arrows directed outwardly from the perfuse vessels in the inner regions), and diffusive interstitial flux 850 (single large arrow in each case, spaced apart from the perfuse vessels, directed radially inwardly). Convective transvascular flux is significantly enhanced after DEX treatment. The large arrows pointing radially outward 840 and large arrows pointing radially inward 850 represent, respectively, the nanocarrier convective and diffusive flux in the tumor interstitium. The direction of interstitial convective transport of nanocarriers is outward towards the periphery. caused by the IFP gradient, while the direction of interstitial diffusive transport of nanocarriers is inward towards the center, caused by the concentration gradient. The overall interstitial fluxes are significantly greater following DEX treatment. The interstitial fluxes and transvascular fluxes are illustrated based on the global optimization results for 13 nm nanocarrier experiments. The interstitial and transvascular flux arrow lengths are each normalized to their own relevant bases for ease of illustration and should not be compared to one another. Since interstitial fluxes are spatially dependent, the arrows represent spatially-averaged fluxes.

### Example 5: Global Optimization Reveals Dose of Dexamethasone Maximizing Nanocarrier Accumulation

Given that a moderate dose of DEX is superior to no DEX and a high dose of DEX for enhancing transvascular transport, there is an optimal dose of DEX that can maximize nanocarrier or antibody concentration accumulation. DEX as a drug for TME normalization is both (1) an antiangiogenic agent that can normalize tumor vessels and (2) a cancer-associated fibroblast reprogramming agent that reduces ECM levels leading to decompressed tumor vessels. The functions of (1) and (2) are associated with vascular hydraulic conductivity Lₚ and interstitial hydraulic conductivity K, respectively. Both Lₚ and K become more favorable for drug delivery with a moderate dose of DEX treatment, but the relative contributions of (1) and (2) cannot be directly controlled with a drug like DEX that affects both. In addition, as indicated by Table 7, an excess dose of DEX decreases Lp thereby limiting transvascular flux for drug delivery. Thus, it is not clear what dose of DEX should be used to maximize the therapeutic effect of a subsequently administered nanocarrier or antibody. The global optimization method and TME-normalizing therapy design formulation (introduced above, with the disclosure directed to TME-Normalizing Therapy Design for Dose Selection) enable the capability to seek the optimal dose of DEX maximizing the nanocarrier accumulation.

As described above, with the disclosure directed to TME-Normalizing Therapy Design for Dose Selection, two cases of TME-normalizing therapy design problems are disclosed: (Case 1) the relationships between DEX dose and Lₚ and K are established based on the original data published previously, expressed as (5) and (6); and (Case 2) the relationships between DEX dose and Lₚ and K are established based on the original data combined with auxiliary data points, expressed as (7) and (8). Both TME-normalizing therapy design problems were solved to global optimality. It took 2.5 h to solve Case 1 and 3.6 h to solve Case 2. The more complicated relationship between DEX dose and hydraulic conductivities in Case 2 resulted in higher complexity and a longer solution time to reach global optimality. Nevertheless, the proposed methodology with a mixed IA/AA approach for the bounding routine was able to locate an optimal solution in hours. Thus, this short computation time demonstrates that the proposed TME-normalizing therapy design methodology is practical for real-world clinical studies. An optimal solution for Case 1 is found at x*=5.30 mg/kg, and for Case 2 is found at x*=4.41 mg/kg. The optimal dose found in Case 1 results in 3% higher concentration accumulation than 3 mg/kg DEX treatment and 74% higher than 30 mg/kg DEX treatment. As a result, the TME-normalizing therapy design methods in this work demonstrate that global optimization can be used in a reasonable time window to determine the optimal dose of DEX, which is predicted to perform 3% better than the best dose determined by the experiments.

### Example 6: Dexamethasone Dose Affects the Transvascular Convective Transport Size-Dependentl

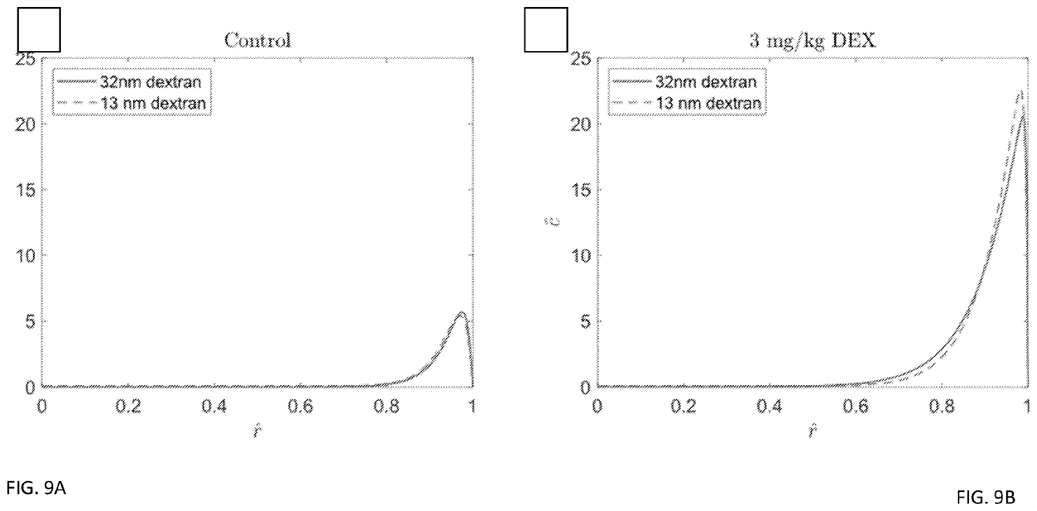

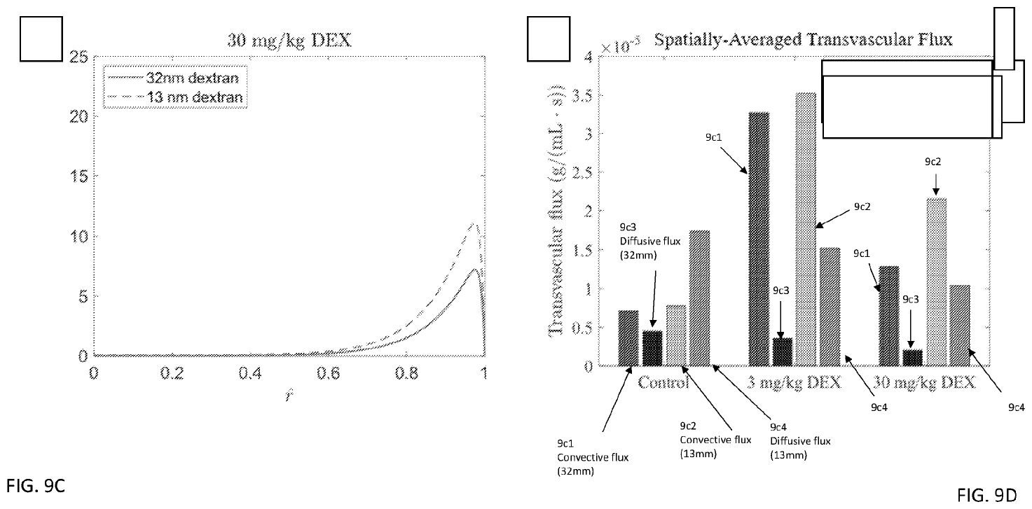

FIGS. 9A-9D show interstitial concentrations of different sizes nanocarriers (500 kDa nanocarrier-32 nm; 70 kDa nanocarrier-13 nm; Case 1-16.40 nm) one-hour post-administration are plotted versus dimensionless tumor radial position r for control in FIG. 9A; 3 mg/kg DEX treatment in FIG. 9B; and 30 mg/kg DEX treatment cases in FIG. 9C. In FIG. 9D, the spatially-average transvascular convective fluxes are plotted for 32 nm and 13 nm dextrans 9c1, 9c2 at one-hour post-administration, and diffusive fluxes are plotted for 32 nm and 13 nm dextrans 9c3, 9c4 at one-hour post-administration. The interstitial concentration with 30 mg/kg DEX treatment for 32 nm dextran is lower than 13 nm dextran mainly due to its lower convective flux.

After finding that the optimal dose of DEX treatment maximizing concentration accumulation, the size of nanocarriers also affects interstitial concentration. Vascular permeability experimental data of two nanocarriers with different hydrodynamic diameters (with data published previously) is compared, because unrelated previous studies demonstrated that vascular permeability depends on the nanocarrier size. The smaller nanocarrier is 13 nm. which is similar to the size of nanoparticle albumin-bound paclitaxel in circulation, and the larger is similar to the size of NC-6004, which is a clinical-stage polymeric micelle containing cisplatin. Using this experimental data and the disclosed mathematical model. the model-predicted interstitial concentration with respect to tumor radial position for these nanocarriers is determined. As illustrated in FIG. 9A, the interstitial concentrations for the control case are almost the same for 32 nm and 13 nm dextrans. And for 3 mg/kg DEX treatment case illustrated in FIG. 9B. the peak for 13 nm dextran is slightly higher, but the overall concentration distribution is still very close for these dextrans. However, for 30 mg/kg DEX treatment case illustrated in FIG. 9C, the concentration profile for 13 nm dextran is higher than 32 nm. A possible reason is that the vessel wall pore size decreases with 30 mg/kg DEX treatment. Thus, the steric hindrance is larger, especially for larger nanocarriers. Consequently, there are fewer larger nanocarriers that transport into the tumor tissue, leading to a lower concentration profile. To better understand this phenomena. the effects of convective and diffusive transport are determined.

The spatially-averaged convective and diffusive fluxes are quantified for 13 nm and 32 nm dextrans to demonstrate their impact on accumulation. As illustrated in FIG. 9D. 3 mg/kg DEX treatment enhances convection to a similar extent for each dextran (360% increase for both 13 and 32 nm dextran compared with the control case). However, 30 mg/kg DEX treatment leads to a 80% increase of convection for 32 nm dextran and a 180% increase for 13 nm dextran. Therefore, the relatively lower convective flux with 30 mg/kg DEX for 32 nm dextran results in less accumulation into the tumor tissue. In addition, DEX reduces diffusion for both nanocarriers. The higher interstitial concentration and smaller pore size with 30 mg/kg DEX lead to lower diffusive fluxes. Since diffusion is inversely related to hydrodynamic diameter of nanocarriers, reduced diffusion after DEX is more important for smaller nanocarriers, which rely on diffusion. In conclusion, 3 mg/kg DEX enhanced transvascular transport size-independently, which conforms to the findings using the ECM normalizing agent tranilast in the art. However, given the antiangiogenic properties of DEX, an excess dose of DEX is less effective for enhancing convection especially for larger nanocarriers.

### Example 7: Global Optimization Determines the Dexamethasone Dose and Nanocarrier Size Maximizing Accumulation

DEX enhances convection yet reduces diffusion, so the optimal hydrodynamic diameter of nanocarrier is determined that exploits the balance of these two effects to realize a maximum accumulation with safety/performance specifications. Three cases of drug size design problems are disclosed. These corresponded to 3 mg/kg DEX treatment, the optimal dose of DEX for Case 1 (5.30 mg/kg), and the optimal dose of DEX for Case 2 (4.41 mg/kg), respectively. The 3 mg/kg dose induced the highest transvascular flux in experiments, whereas Case 1 and Case 2 were determined from the corresponding TME-normalizing therapy design problems. These drug size design problems formulated as (10) were solved to global optimality. Theoptimal solutions found and time costs for each case are summarized in Table 10. Optimal nanocarrier sizes in these designs strictly satisfy the safety/performance requirements to avoid potential side effects and guarantee the effectiveness, which constrain the nanocarrier concentrations in the periphery of tumor normal tissue, as demonstrated in (10). Though smaller nanocarriers diffuse and accumulate inside the tumor interstitial space more quickly, it might violate the safety specifications in these designs. Thus, these optimal solutions account for the drug size design results with requirements. In addition, these problems can be solved in minutes, demonstrating the practicability for real-world applications.

Table 10 shows optimal solutions and time costs of drug size design problems for the case studies of 3 mg/kg DEX treatment, Case 1, and Case 2 of the therapy design problem.

TABLE 10

| Case study | 3 mg/kg | Case 1 | Case 2 |
|---|---|---|---|
| Optimal solution (d_m\hat{ } *, nm) | 19.65 | 16.55 | 12.51 |
| Time (s) | 355 | 384 | 120 |

The simultaneous therapy design approach with ANN models formulated as (12) was also performed with Case 1 and Case 2 studies. An optimal solution for Case 1 was found at

$$( x * , {d}_{m}^{*} ) = ( 5.32 , 16.4 ) and for Case 2 at ( x * , {d}_{m}^{*} ) = ( 4.36 , 12.41 ) .$$

The time costs are 42 s for Case 1 and 350 s for Case 2. However, global optimal solution of (11) with the mechanistic model could not be obtained within a reasonable time limit. Continued research on global bounding methods may be able to accelerate convergence and address this issue in the future. Alternatively. a multi-start local optimization procedure is implemented for problems formulated as (11) and selected the results with the lowest objective function values:

$$( ( x * , {d}_{m}^{*} ) = ( 5.33 , 16.54 ) for Case 1 and ( x * , {d}_{m}^{*} ) = ( 4.36 , 12.58 ) for Case 2 ) .$$

Optimal solutions obtained with the ANN models are very close to the best-found local optimal results obtained via the multi-start procedure. Since the ANN models were very accurate surrogates of the mechanistic model, this provides supporting evidence that the local results obtained are close estimates of the global optima. Therefore, these results provide support for the practicability of the ANN models for use in optimal decision-making in cancer therapeutics.

The therapy design methods in this work provide capability to identify optimal dose and drug size for maximizing the improvement in nanocarrier delivery induced by TME-normalizing therapies.

Rigorous methods of model validations and optimal TME-normalizing dose and nanocarrier size therapy designs were developed. This work was motivated by the need for more rigorous methods for in silico model-based decision-making in cancer research. Use of a comprehensive theoretical framework for model-based applications in preclinical PKPD research and development. The dynamic optimization problems were formulated as PDE-constrained NLPs and solved to global optimality, providing rigorous solutions for cancer drug delivery studies. An efficient bounding routine using IA/AA and DI approaches and a special bounding rule for the Péclet number in the solute source term were proposed for improving the performance of the global optimization algorithm. In addition, machine learning approaches were utilized to establish a data-driven model via ANNs as surrogate models for the original PDE system. The ANNs were utilized in place of the mechanistic model for solving the parameter estimation problems with a simplified formulation. In particular, based on the global solution values obtained for the hydraulic conductivities, transvascular transport was quantified with respect to convective and diffusive fluxes to elucidate their contributions to the accumulation of anticancer nanocarriers in tumors following TME-normalizing DEX treatment. Moreover, a methodology for optimal TME-normalizing therapy design was proposed to optimize the dose of DEX for enhanced accumulation of anticancer nanocarriers in tumors. The nanocarrier size design method was also proposed to determine an optimal size for patient-specific TMEs with safety/performance specifications. Finally, a simultaneous design formulation was considered to determine an optimal dose of DEX and an optimal nanocarrier size that would lead to maximized accumulation in the tumor interstitium.

### Supplementary Example 1: Tumor Transport Model

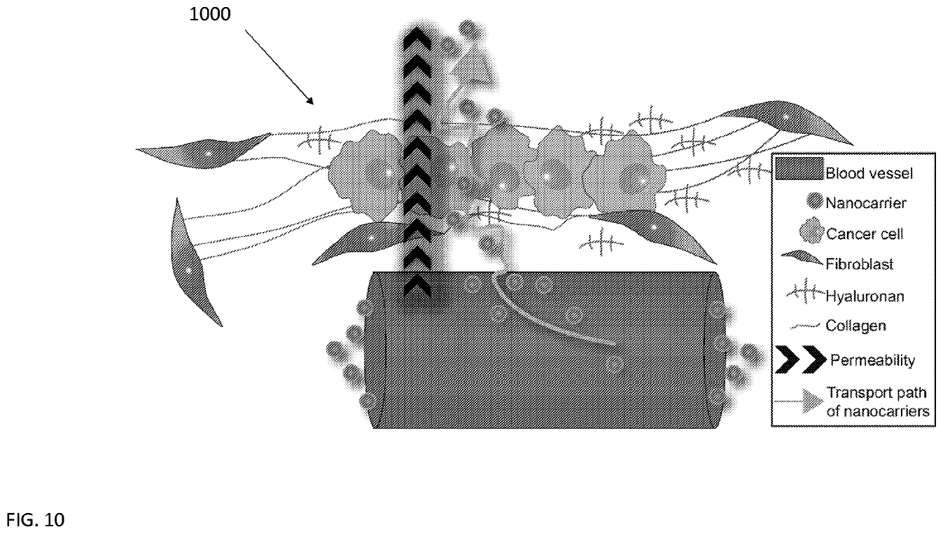

The 1-dimensional (1D) tumor transport model proposed previously is used as a mechanistic foundation for determining transvascular exchange and extravascular transport in tumors. The real vasculature of the tumor is intricate and the cells between regions have large differences. There is a necrotic region at the center of the tumor (i.e., most/all cells are dead). In contrast, the outer region of the tumor contains rapidly dividing cells requiring a large blood supply by abundant active blood vessels. Thus, actual solid tumors are spatially heterogeneous and it may be that some physiological parameters in this model are spatially dependent. Tumor microenvironment (TME) is simplified to be spatially homogeneous without lymphatics or extravascular bindings, which is helpful for certifying and evaluating the overall role of the interstitial fluid pressure (IFP) on fluid transport and penetration of nanocarriers in a tumor. The blood vessels, cells, extracellular matrix (ECM), and other microscopic structures, as illustrated in FIG. 10 (discussed below), are also not considered explicitly in the model because this level of granularity is not important at the length scales concerned. In addition, one focus is determining the overall macromolecular solute concentrations in a tumor over a prescribed time horizon. Therefore, spatial averaging is utilized in the data and simulation results, which essentially homogenizes the macroscopic structures. In addition, it is also assumed that the vasculature is distributed continuously over the spatial domain rather than at discrete or localized positions.

FIG. 10 shows a diagram 1000 of the tumor microenvironment illustrating fluid and solute transport from the blood vessels to the interstitium with high transvascular permeability.

### Supplementary Example 1.1: Fluid Transport

The fluid transport in the interstitium of a tumor follows Darcy's law:

$$u = - K ∇ p . \qquad ( S1 )$$

Here, u is the interstitial fluid velocity (IFV) (cm/s), K is the hydraulic conductivity of tumor interstitium (cm²/mm Hg-sec), and p is the IFP (mm Hg). With axisymmetric flow in the spherical coordinate, (S1) can be simplified to

$$u = - K \frac{dp}{dr} . \qquad ( S2 )$$

where r is the radial position (cm).

The continuity equation for steady-state incompressible fluid flow in spherical coordinates is given by:

$$\frac{1}{{r}^{2}} \frac{d ( {r}^{2} u )}{dr} = {L}_{p} \frac{S}{V} ( {p}_{v} - p ) . \qquad ( S3 )$$

Here, Lₚ is the hydraulic conductivity of the microvascular wall (cm/mm Hg-sec), S/V is the vascular surface area per unit volume (cm⁻¹), and pᵥ is the vascular pressure (mm Hg). Substituting (S1) into the continuity equation (S3), the steady-state fluid transport model is given by

$$\frac{1}{{r}^{2}} \frac{d}{dr} ( {r}^{2} \frac{dp}{dr} ) = \frac{{α}^{2}}{{R}^{2}} ( p - {p}_{ss} ) , \\ Where \\ α = R \sqrt{\frac{S}{V} \frac{{L}_{p}}{K}} \qquad ( S4 )$$

is a dimensionless parameter representing the ratio of resistances of the fluid flow in the interstitium to across the vasculature, R is the radius of the spherical tumor (cm), and pₛₛ is the steady-state interstitial pressure where the efflux from the vessels equals the influx (mm Hg), and is equal to pᵥ.

The boundary conditions consist of a no-flux symmetry condition at the center of the spherical tumor and a Dirichlet condition at the periphery, respectively, as

$$\frac{dp}{dr} {\vert}_{r = 0} = 0 , \\ P {\vert}_{r = R} = {p}_{∞} . \qquad ( S5 )$$

where p_{∞} denotes the surrounding tissue pressure (mm Hg).

### Supplementary Example 1.2: Solute Transport

To describe and characterize the transport mechanism of nanocarriers in tumors, the macromolecular solute transport model is governed by the convection-diffusion equation:

$$\frac{\partial c}{\partial t} + \frac{1}{{r}^{2}} \frac{\partial ( {r}^{2} uc )}{\partial r} = D \frac{1}{{r}^{2}} \frac{\partial }{\partial r} ( {r}^{2} \frac{\partial c}{\partial r} ) + {ϕ}_{s} , \qquad ( S6 )$$

where c is the concentration of the solute in the interstitium of the tumor (g/mL), D is the diffusion coefficient (cm²/sec), and ϕₛ is the distributed source term based on a vessel pore model for transcapillary exchange, given by

$${ϕ}_{s} = {L}_{p} \frac{S}{V} ( {p}_{v} - p ) ( 1 - σ ) {c}_{v} + P \frac{S}{V} ( {c}_{v} - c ) \frac{Pe}{{e}^{Pe} - 1} \qquad ( S7 )$$

Here, Pe=Lₚ(pᵥ−p)(1−σ)/P is the Péclet number representing the ratio of the rates of convection to diffusion across the vascular wall, σ is the solute reflection coefficient, P is the vascular permeability of the solute through the vascular wall (cm/sec), and [symbol] ᵥ is the solute concentration in tissue vessels (g/mL). Since the bolus injection model is applied, the vascular solute concentration decays exponentially with time as [symbol] ᵥ= [symbol] ₒ [symbol] ^{-t/k}^{_{d}}, where [symbol] ₀ is the initial macromolecular solute concentration in the blood (g/mL), and k_{d} is the half-life circulation time of the nanocarriers (sec).

It is assumed that no macromolecular solutes exist in the tumor before injection, and therefore the initial condition is [symbol] (0, r)=0. The boundary conditions are defined as:

$$\begin{matrix}- D \frac{\partial c}{\partial r} {\vert}_{r = 0} + uc {\vert}_{r = 0} = 0 \\ c {\vert}_{r = R} = {c}_{∞}\end{matrix} \qquad ( S8 )$$

where the interstitial concentration satisfies the no-flux condition at the center, is continuous across the tumor periphery, and equals [symbol] _{∞}, representing the concentration (g/mL) in the normal tissue surrounding the tumor.

### Supplementary Example 1.3: Pore Theory

Applying the pore theory developed in the art, pores of the vessels are assumed to be cylindrical, in this case, the hydraulic conductivity is determined of the tumor vessels Lₚ, the vascular permeability P, and the reflection coefficient σ by the pore theory

$${L}_{p} = \frac{γ {r}_{o}^{2}}{8 μ L} \\ P = \frac{γ {HD}_{o}}{L} \\ {D}_{o} = \frac{{k}_{B} T}{6 n μ {r}_{m}} \\ σ = 1 - W \qquad ( S9 )$$

where γ is the fraction of the surface area occupied by pores, rₒ is the pore radius (nm), μ is the blood viscosity (mm Hg-sec), L is the thickness of the vessel wall (μm), Dₒ is the diffusion coefficient of the nanocarrier in free solution at 37° C. given by the Stokes-Einstein relationship, k_{B} is the Boltzmann constant (1.380648×10⁻²³ J/K), T is the temperature of solution (310.15 K), rₘ is the particle radius (nm), H and W are respectively diffusive and convective hindrance factors based on the ratio of the particle size over the pore size, which are given in the art:

$$H = \frac{6 n Φ}{{K}_{t}} , \\ W = \frac{Φ ( 2 - Φ ) {K}_{s}}{2 {K}_{t}} , \qquad ( S10 )$$

where Φ is the partition coefficient defined as the ratio of the average intrapore concentration to that in the bulk solution at equilibrium. When the interactions between the solutes and pore wall are purely steric, the partition coefficient is taken as Φ=(1−λ)², where λ is the ratio of particle size (dₘ, nm) to the pore size (dₒ, nm). The value of λ should be less than one. The Kₜ and Kₛ factors for the convective hindrance term W are defined as:

$$\begin{matrix}{K}_{t} = \frac{9}{4} {π}^{2} \sqrt{2} {( 1 - λ )}^{- 5 / 2} [ 1 + {\sum }_{k = 1}^{2} {{α}_{k} ( 1 - λ )}^{k} ] + {\sum }_{k = 0}^{4} {α}_{k + 3} {λ}^{k} \\ {K}_{s} = \frac{9}{4} {π}^{2} \sqrt{2} {( 1 - λ )}^{- 5 / 2} [ 1 + {\sum }_{k = 1}^{2} {{β}_{k} ( 1 - λ )}^{k} ] + {\sum }_{k = 0}^{4} {β}_{k - 3} {λ}^{k}\end{matrix} \qquad ( S11 )$$

The corresponding coefficients aₖ and bₖ are listed in Table 11. As indicated by (S9), the vascular permeability P depends on the particle size and vessel wall properties, such as pore size, thickness, charge, and arrangement. Larger particles will result in lower P, and when the particle size is larger than the pore cut-off size, P becomes zero. The vascular hydraulic conductivity Lₚ relies on the morphology of the wall and the fraction of the wall surface occupied by active pores.

Table 11 shows hydrodynamic coefficients used for the cylindrical pore model.

TABLE 11

| k | 1 | 2 | 3 | 4 | 5 | 6 | 7 |
|---|---|---|---|---|---|---|---|
| α_k | −73/60 | 77293/50400 | −22.5083 | −5.6117 | −0.3363 | −1.216 | 1.647 |
| β_k | 7/60 | −2227/50400 | 4.0180 | −3.9788 | −1.9215 | 4.392 | 5.006 |

### Supplementary Example 1.4

The fluid and solute transport models were solved numerically. First, the dimensionless form of the tumor radius, IFP, and solute concentration, were defined as:

$$\hat{r} = \frac{r}{R} . \\ \hat{p} = \frac{p - {p}_{∞}}{{p}_{ss} - {p}_{∞}} . \\ \hat{c} = \frac{c - {c}_{∞}}{{c}_{o} - {c}_{∞}} . \qquad ( S12 )$$

After reformulating the tumor transport model into dimensionless form, the centered finite difference method was used to discretize the spatial domain. The IFP profile is obtained by solving the fluid transport model (S4). As for the solute transport model, the backward difference scheme was employed for discretization of the first partial derivative ∂c/∂r. Then, the explicit Euler method was used to integrate the transient convection-diffusion equation with step-size set as h=15 s to obtain the medicine concentration profile over the tumor radius.

### Supplementary Example 2: Set-Valued Mapping Approaches

### Supplementary Example 2.1: Interval Arithmetic and Affine Arithmetic

Interval arithmetic (IA) is an arithmetic performed on intervals according to primitive interval computation rules. The main objective of IA to calculate upper and lower bounds for the range of a function in one or more variables. IA suffers from the dependency problem as the different intervals in an equation are treated as entirely independent variables. When some of the intervals depend on each other (e.g., a variable occurs several times in an equation), the combinations of IA operations of the function may significantly overestimate the enclosure of the function.

Affine Arithmetic (AA) can overcome the overestimation induced by the dependency problem of traditional IA. AA keeps track of the dependency between the interval variables throughout the calculations resulting in better interval approximations in most cases. In addition, the associated properties for the joint range of the interval variables can be represented as a geometry by AA that reduces overestimation. When implementing none-affine operations, an extra noise term is required to estimate the affine approximations of the non-affine part for each operation. Generally speaking, this results in the elementary operations of AA to be more computationally expensive than standard IA. Non-affine operations and the additional complexity that they introduce, are ignored, resulting in no extra time cost over standard IA.

### Supplementary Example 2.2

Differential Inequalities (DI) is an approach using IA for constructing the component-wise lower and upper bounds on the solution set of a system of ordinary differential equations (ODEs). The DI methods can be categorized into two types: continuous-time DI and discrete-time DI. In continuous-time DI, an auxiliary system of ODE-IVPs is formulated and directly sent to a numerical integrator for constructing the bounds. In contrast, the discrete-time DI reformulates the system of ODEs into a discrete-time form. Then the bounding rules are applied at each discrete time point. Discrete-time DI method was utilized in this work. In addition, for systems in which additional bounding information is known a priori, an interval refinement operator can be applied to the standard DI for further reducing overestimation of the bounding results.

### Supplementary Example 3: Simplification Of Inequality Constraints

Inequality constraints on the superficial IFP can be expressed as linear constraints on the optimization variables, Lp and K, such that K=ζLₚ, with ζ∈ᵢ. First, the dimensionless analytical solution of the fluid transport model (S4) can be derived as:

$$\hat{p} = ( 1 - \frac{sinh ( \hat{r} α )}{\hat{r} sinh ( α )} ) , \qquad ( S13 )$$

where a is given in (S4).

Then, the IFP in the superficial region can be represented as:

$${\hat{p}}_{peri} = ( 1 - \frac{sinh ( {\hat{r}}_{peri} α )}{{\hat{r}}_{peri} sinh ( α )} ) , \qquad ( S14 )$$

where r [symbol] ₚₑᵣᵢ is the dimensionless radius from the center towards the superficial region of a tumor. Substituting (S14) into the inequality constraints of (4) results in:

$$( 1 - \frac{sinh ( {\hat{r}}_{peri} α )}{{\hat{r}}_{peri} sinh ( α )} ) \geq {\hat{p}}_{peri , m i n} . \qquad ( S15 )$$

$$( 1 - \frac{sinh ( {\hat{r}}_{peri} α )}{{\hat{r}}_{peri} sinh ( α )} ) \leq {\hat{p}}_{peri , ma x} . \qquad ( S16 )$$

If (S15) is active, then the following equality holds:

$$1 - \frac{sinh ( {\hat{r}}_{peri} α )}{{\hat{r}}_{peri} sinh ( α )} \leq {\hat{p}}_{peri , m i n} . \qquad ( S17 )$$

Differentiating (S17) with respect to Lₚ, yields the following expression:

$$( - \frac{{\hat{r}}_{peri} sinh ( α ) cosh ( {\hat{r}}_{peri} α ) - sinh ( {\hat{r}}_{peri} α ) cosh ( α )}{{\hat{r}}_{peri} {sinh}^{2} ( α )} ) ( \frac{d α}{d {L}_{p}} ) = 0. \qquad ( S18 )$$

Since a>0 always holds (L_{p>0)} it can be verified that

$$( - \frac{{\hat{r}}_{peri} sinh ( α ) cosh ( {\hat{r}}_{peri} α ) - sinh ( {\hat{r}}_{peri} α ) cosh ( α )}{{\hat{r}}_{peri} {sinh}^{2} ( α )} ) \neq 0.$$

Therefore, if the constraint is active, then:

$$\frac{d α}{d {L}_{p}} = 0 ⇒ \frac{d ( R \sqrt{\frac{{SL}_{p}}{\sqrt{K}}} )}{d {L}_{p}} = 0 \qquad ( S19 )$$

For this expression to hold, this means that α must be constant with respect to Lₚ. Since all parameters in α other than Lₚ and K are constants, K must necessarily be a scalar multiple of Lₚ.

This gives the following result:

$$K = ζ {L}_{p} , for some ζ ∈ i such that \hat{p} = {\hat{p}}_{peri , m i n} . \qquad ( S20 )$$

By the same procedure, K must be a scalar multiple of Lₚ if (S16) is active. Therefore, (S15) and (S16) can be simplified as, respectively:

$$K \leq {ζ}_{m ax} {L}_{p} , \qquad ( S21 )$$

$$K \geq {ζ}_{m i n} {L}_{p} . \qquad ( S22 )$$

The values of ζmin and ζₘₐₓ are listed in Table 5. The values for (min are calculated according to the following procedure:
1. Choose two different values of Lₚ within the interval bounds.
2. Solve the nonlinear equation (S17) with each value of Lₚ for the corresponding K values.
3. Compute ζₘᵢₙ as the slope of the secant line joining the two points on an Lₚ versus K plot.
4. The calculation of ζₘₐₓ values follow analogously.

### Supplementary Example 4: Relationship Between Nanocarrier Size and Physiological Parameters

There are two physiological parameters directly related to nanocarrier size dₘ: diffusion coefficient D and half-life circulation time k_{d}. The previous experimental results for their correlations are listed in Table 12.

In Table 12, data for diffusion coefficients and blood half-life circulation time with respect to nanocarrier sizes are reported.

TABLE 12

| Particle size | 12 nm | 60 nm | 125 nm | 250 nm |
|---|---|---|---|---|
| Diffusion coefficient | 2 × | 5 × | 6 × | 1 × |
| (cm²/s) | 10\hat{ }(−7) | 10\hat{ }(−8) | 10\hat{ }(−9) | 10\hat{ }(−9) |
| Half-life circulation | 1480 | 995 | 582 | 500\hat{ } * |
| time (min) | | | | |

Nonlinear regression models are established (power model for D versus dₘ; Gaussian model for k_{d} versus dₘ) for these quantities as:

$${f}_{D} = 1.981 \times {10}^{- 6} \cdot {d}_{m}^{- 1.157} + 2.221 \times {10}^{- 8} . \\ {f}_{{k}_{d}} ( {d}_{m} ) = 1081 exp ( - {( \frac{{d}_{m} + 16.63}{84.82} )}^{2} ) + 517.4 exp ( - {( \frac{{d}_{m} - 65.61}{996.6} )}^{2} ) . \qquad ( S23 )$$

where f_{D} and fₖ_{_{d}} represent the values of D and k_{d}, respectively.

According to one or more embodiments, as indicated above, a personalized method of treating a cancer patient is disclosed, the method including administering to the cancer patient a first dose of an adjuvant to chemotherapy (dexamethasone), performing in vivo imaging to determine the effective permeability (Peff) of the first dose of dexamethasone in a tumor in the cancer patient, executing the process disclosed herein, determining a second dose of the adjuvant to chemotherapy and a nanocarrier size for a chemotherapy drug for the cancer patient, and optionally administering the second dose of the adjuvant to chemotherapy and the chemotherapy drug in a sized nanocarrier.

Dexamethasone is an adjuvant to chemotherapy which is used to reduce inflammation and suppress the body's immune response. Dexamethasone can be administered before, during, or after chemotherapy, typically as an oral formulation.

Typical doses of dexamethasone are 0.3 to 1.7 mg/kg/day.

In vivo imaging to determine the effective permeability (Peff) of the first dose of dexamethasone in a tumor in the cancer patient can include microscopy techniques such as computed tomography (CT), magnetic resonance imaging (MRI) ultrasound (US), positron emission tomography (PET), single-photon emission computed tomography (SPECT), fluorescence reflectance imaging (FRI), fluorescence-mediated tomography (FMT) bioluminescence imaging (BLI), laser-scanning confocal microscopy (LSCM), multiphoton microscopy (MPM), and the like.

Exemplary nanocarriers have diameters of 5 to 500 kDa.

The type of nanocarrier is not limited and includes polymeric nanoparticles, protein-based carriers such as cell surface proteins, micelles, dendrimers, lipid nanoparticles including liposomes, inorganic nanoparticles, magnetic nanoparticles, and the like. Polymeric nanoparticles include natural polymers such as albumin, heparin and chitosan as well as biodegradable polymers such as PLA, PLC and PLGA. Inorganic nanoparticles include mesoporous silica NCs (MSNCs), gold NCs (AuNCs) [21], magnetic NCs (MNCs), carbon nanotube NCs (CNT-NCs), graphene oxide and quantum dots (QDs).

The type of chemotherapy will depend on the type of tumor to be treated. Exemplary chemotherapies include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide. carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflornithine, elsamitrucin, enloplatin, enpromate, epipropidine, epirubicin. erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, losoxantronc. masoprocol, maytansine, mechlorethamine hydrochloride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, olaparib, ormnaplatin, oxaliplatin, oxisuran, paclitaxel, pegaspargase, peliomycin, pentamustine, peplomycin, perfosfamide, pipobroman, piposulfan, piroxantrone hydrochloride, plicamycin, plomestane, porfimer. porfiromycin, prednimustine, procarbazine, puromycin, pyrazofurin, riboprine, rogletimide, rucaparib, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, velaparib, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, zorubicin.

Thus, according to an aspect of the disclosure, herein disclosed is a personalized method of treating a cancer patient with a tumor utilizing a computing system comprising a processing system and a memory system storing instructions that are executed by the processing system. the method comprising the computing system performing steps of: performing parameter estimation to determine physiological parameters π of the tumor, including vascular hydraulic conductivity and interstitial hydraulic conductivity; determining whether the selected tumor transport model is valid or invalid by solving for physiological parameters π, and upon determining that the selected tumor transport model is valid, the method includes determining a treatment; and the method further includes: applying the treatment to a cancer patient.

According to an additional aspect of the disclosure, directed to the method, the step of performing parameter estimation to determine physiological parameters π includes: measuring Peff and utilizing a parameter estimation problem to predict/determine K and L, where Peff is an effective permeability quantified as a rate of fluorescent signal passing through tumor vessel walls; Lp is a hydraulic conductivity of a microvascular wall (cm/mm Hg-sec); K is a hydraulic conductivity of tumor interstitium (cm2/mmHg-sec); or measuring K directly and utilizing the experimental data when solving the parameter estimation problem.

According to an additional aspect of the disclosure, directed to the method, the vascular density S/V is measured when measuring Peff vascular density S/V is measured when measuring Peff, where S/V is measured as vascular surface area per unit volume (cm-1).

According to an additional aspect of the disclosure, directed to the method, the step of performing parameter estimation includes: determining a time-dependent spatially-averaged drug concentration profile c_{avg}^{data}, representing a state of a tumor, including an ability of drugs and nutrients to accumulate in the tumor.

According to an additional aspect of the disclosure, directed to the method, the step of performing parameter estimation includes: determining the time-dependent spatially-averaged drug concentration profile c_{avg}^{data} by utilizing: data

$$\frac{{dc}_{avg}^{data}}{d t} = {P}_{eff} \frac{S}{V} ( {c}_{v} - {c}_{avg}^{data} )$$

where cᵥ is a solute concentration in vessels of a tumor (g/mL) and S/V is a vascular surface area per unit volume (cm⁻¹).

According to an additional aspect of the disclosure, directed to the method, the step of performing parameter estimation includes: determining the physiological parameters n via a first parameter estimation problem:

$${min}_{π ∈ \prod } {\sum }_{i = 1}^{n} {( {\hat{c}}_{avg} ( {t}_{i} , π , {d}_{m} ) - {\hat{c}}_{avg}^{{}_{data}} ( {t}_{i} ) )}^{2} \\ s . t . {\hat{p}}_{peri} ( π ) \leq {\hat{p}}_{peri , max} \\ {\hat{p}}_{peri} ( π ) \geq {\hat{p}}_{peri , min} ,$$

where ĉ_{avg} is a dimensionless spatially-averaged concentration of solute that is determined by averaging a dimensionless concentration ĉ for all spatial nodes from a mechanistic solute transport model that is utilized as a parametric model output;

π=(Lₚ, K)∈Π⊂ [symbol] ⁿ^{_{π}}

represents a vector of physiological parameters of the spatially-averaged solute transport model; Lp being a hydraulic conductivity of a microvascular wall (cm/mm Hg-sec); K is a hydraulic conductivity of tumor interstitium (cm2/mmHg-sec); and dₘ is a diameter of a nanoscale (nm) biomolecule or macromolecular medicine.

According to an additional aspect of the disclosure, directed to the method, upon determining that the model is invalid for this dataset, the method includes the computing system; obtaining more data and solving the parameter estimation problem again; or selecting a different tumor transport model, or modifying the tumor transport model, and solving the parameter estimation problem.

According to an additional aspect of the disclosure, directed to the method, utilizing the experimental data to model spatial-temporal transport in tumors includes the computing system performing the steps of: utilizing a mechanistic tumor transport model directly; or utilizing the mechanistic tumor transport model to generate simulation data to train a machine learning model.

According to an additional aspect of the disclosure, directed to the method, when repeating the step of performing parameter estimation, computing system performs the step of: utilizes the first parameter estimation problem when determining the physiological parameters π with the mechanistic tumor transport model; or utilizes a second parameter estimation problem when determining the physiological parameters π with the data-driven tumor transport model, the second parameter estimation problem being:

$${min}_{π ∈ \prod } {\sum }_{i = 1}^{n} {( {\hat{c}}_{avg , i}^{{}_{ANN}} ( π ) - {\hat{c}}_{avg}^{{}_{data}} ( {t}_{i} ) )}^{2} \\ s . t . {\hat{p}}_{peri} ( π ) \leq {\hat{p}}_{peri , max} \\ {\hat{p}}_{peri} ( π ) \geq {\hat{p}}_{peri , min} ,$$

where ĉ^{ANN}_{avg} represents a dimensionless spatial average nanocarrier concentration at discrete time node i calculated from an ANN surrogate model, and utilizing the experimental data when solving the parameter estimation problems.

According to an additional aspect of the disclosure, directed to the method, applying the treatment includes the computing system performing steps of: determining a dose selection if the patient has not yet received adjunct therapy, where dose selection refers to the TME-normalizing agent, which is an adjunct; or determining a drug-size selection if the patient has received adjunct therapy, where the drug-size selection refers to the anticancer drug, which is differentiated from the adjunct, which is the TME-normalizing agent.

According to an additional aspect of the disclosure, directed to the method, determining the dose selection includes the computing system performing steps of: determining vascular hydraulic conductivity Lp and interstitial hydraulic conductivity K from empirical correlations between a cause-and-effect relationship between a tumor-normalizing dose, K and Lp; and determining an optimal dose that maximizes a drug accumulation in tumors via determining:

$${max}_{x ∈ X} {\hat{c}}_{avg}^{} ( {t}_{f} , ( {f}_{{L}_{p}}^{j} ( x ) , {f}_{K}^{j} ( x ) ) , {d}_{m} )$$

with j∈{r, p}, where t_{f} is a final time, x is a TME-normalization regimen dose, and f_{Lp} and fₖ^{j} represent Lₚ and K, respectively, following treatment with the regimen dose, obtained from the experimental data.

According to an additional aspect of the disclosure, directed to the method, executing the drug-size includes the computing system performing step of: determining an optimal size dₘ of the anticancer nanocarrier by determining

$${max}_{{d}_{m} ∈ Z} {\hat{c}}_{avg} ( {t}_{f} , π , {d}_{m} ) \\ s . t . {\hat{c}}_{peri} ( {t}_{f} , π , {d}_{m} ) \leq {λ}_{1} \\ {\hat{c}}_{peri} ( {t}_{f} , π , {d}_{m} ) \geq {λ}_{2} .$$

where λ₁ is a threshold for a safety constraint and λ₂ is a performance constraint.

According to an additional aspect of the disclosure, directed to the method, determining the treatment includes the computing system performing step of simultaneously determining the dose selection and the drug-size.

According to an additional aspect of the disclosure, directed to the method, determining the treatment includes the computing system performing step of applying empirical correlations that relate tumor physiology to adjunct dose.

According to an additional aspect of the disclosure, directed to the method, the machine learning model is utilized, and determining the treatment includes the computing system performing steps of: determining vascular hydraulic conductivity Lₚ and interstitial hydraulic conductivity K from empirical correlations between a cause-and-effect relationship between a tumor-normalizing dose, K and Lp, and determining

$${max}_{x ∈ X , {d}_{m} ∈ Z} {\hat{c}}_{avg} ( {t}_{f} , ( {f}_{{L}_{p}}^{j} ( x ) , {f}_{K}^{j} ( x ) ) , {d}_{m} ) \\ s . t . {\hat{c}}_{peri} ( {t}_{f} , ( {f}_{{L}_{p}}^{j} ( x ) , {f}_{K}^{j} ( x ) ) , {d}_{m} ) \leq {λ}_{1} \\ {\hat{c}}_{peri} ( {t}_{f} , ( {f}_{{L}_{p}}^{j} ( x ) , {f}_{K}^{j} ( x ) ) , {d}_{m} ) \geq {λ}_{2} .$$

with j∈{r, p}, where t_{f} is a final time, x is a TME-normalization regimen dose, and f_{Lp}^{j} and fₖ^{j} represent Lₚ and K, respectively, following treatment with the dose, obtained from the experimental data, λ₁ is a threshold for a safety constraint and λ₂ is a performance constraint.

According to an additional aspect of the disclosure, directed to the method, the ANN surrogate model is utilized, and determining the treatment includes the computing system performing steps of: determining vascular hydraulic conductivity Lₚ and interstitial hydraulic conductivity K from empirical correlations between a cause-and-effect relationship between a tumor-normalizing dose, K and Lₚ, and determining

$${max}_{x ∈ X , {d}_{m} ∈ Z} {\hat{c}}_{avg}^{{}_{ANN}} ( ( {f}_{{L}_{p}}^{j} ( x ) , {f}_{K}^{j} ( x ) ) , {d}_{m} ) \\ s . t . {\hat{c}}_{peri}^{{}_{ANN}} ( ( {f}_{{L}_{p}}^{j} ( x ) , {f}_{K}^{j} ( x ) ) , {d}_{m} ) \leq {λ}_{1} \\ {\hat{c}}_{peri}^{{}_{ANN}} ( ( {f}_{{L}_{p}}^{j} ( x ) , {f}_{K}^{j} ( x ) ) , {d}_{m} ) \geq {λ}_{2} .$$

with j∈{r, p}, where t_{f} is a final time, x is a TME-normalization regimen dose, and f_{Lp}^{j} and fₖ^{j} represent Lₚ and K, respectively, following treatment with the dose, obtained from the experimental data, λ₁ is a threshold for a safety constraint and λ₂ is a performance constraint.

According to an additional aspect of the disclosure, herein disclosed is a computerized system comprising: a processing system and a memory system storing instructions that are executed by the processing system such that the system is configured to performing steps of: performing parameter estimation to determine physiological parameters π of the tumor, including vascular hydraulic conductivity and interstitial hydraulic conductivity; determining whether the selected tumor transport model is valid or invalid by solving for physiological parameters π, and upon determining that the selected tumor transport model is valid, the method includes determining a treatment; and the method further includes: applying the treatment to a cancer patient.

According to an additional aspect of the disclosure, herein disclosed is a computer program product comprising a memory device having computer executable instructions stored thereon, which when executed by one or more processors cause the one or more processors to perform a plurality of operations comprising: performing parameter estimation to determine physiological parameters π of the tumor, including vascular hydraulic conductivity and interstitial hydraulic conductivity; determining whether the selected tumor transport model is valid or invalid by solving for physiological parameters π, and upon determining that the selected tumor transport model is valid, the method includes determining a treatment; and the method further includes: applying the treatment to a cancer patient.

Turning now to FIG. 11, the figure shows a medical system for training and/or application of a machine-learnt classifier for tumor information. The medical system includes a medical imaging system 111111, a processor 1113, a memory 1115, and a display 1116. The processor 1113 and the memory 1115 are shown separate from the medical imaging system 1111, such associated with being a computer or workstation apart from the medical imaging system 1111. In other embodiments, the processor 1113 and/or memory 1115 are part of the medical imaging system 1111. In alternative embodiments, the medical system is a workstation, computer, or server. For example, the medical imaging system 1111 is not provided or is provided for acquiring data representing a volume. and a separate database, server, workstation, and/or computer is provided for extracting features and applying a classifier to predict one or more results. Additional. different, or fewer components may be used.

The system is used for application of a machine-learnt model (e.g., one or more machine-learnt classifiers). In alternative embodiments, the system is used for training with machine learning and/or generation of the examples in the database. Where only synthetic samples generated from a computational model of a tumor is used, the medical imaging system 1111 may not be provided. Where the samples of the library, even if from actual patients (e.g., scan data representing actual scans), are stored in the memory 1115, the medical imaging system 1111 may not be provided.

The computing components, devices, or machines of the medical system, such as the medical imaging system 1111 and/or the processor 1113 are configured by hardware. software. and/or firmware to perform calculations or other acts. The computing components operate independently or in conjunction with each other to perform any given act, such as the acts of any of the methods described above. The act is performed by one of the computer components, another of the computing components, or a combination of the computing components. Other components may be used or controlled by the computing components to scan or perform other functions.

The medical imaging system 1111 is any now known or later developed modality for scanning a patient. The medical imaging system 1111 scans the patient. For example, a C-arm x-ray system (e.g., DynaCT from Siemens), CT like system, or CT system is used. Other modalities include MR, x-ray, angiography, fluoroscopy, PET, SPECT, or ultrasound. The medical imaging system 1111 is configured to acquire the medical imaging data representing the patient. The scan data is acquired by scanning the patient using transmission by the scanner and/or by receiving signals from the patient.

The memory 1115 is a buffer, cache, RAM, removable media, hard drive, solid state drives, magnetic, optical, database, or other now known or later developed memory. The memory 1115 is a single device or group of two or more devices. The memory 1115 is within the system 1111, part of a computer with the processor 1113, or is outside or remote from other components.

The memory 1115 is configured to store medical scan data, other data, extracted features, examples (e.g., training data or data from other patients), and/or other information. Output results, information derived from the results, or calculations used to determine the results are stored in the memory 1115. The memory 1115 stores one or more matrices for the machine-learnt regression models. The memory can store the learned/trained regression models as well as the mechanistic models.

The memory 1115 is additionally or alternatively a non-transitory computer readable storage medium with processing instructions. The memory 1115 stores data representing instructions executable by the programmed processor 1113. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the FIGS. Or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 1113 is a general processor, digital signal processor, three-dimensional data processor, graphics processing unit, application specific integrated circuit, field programmable gate array, digital circuit, analog circuit, combinations thereof, or other now known or later developed device for processing data. The processor 1113 is a single device, a plurality of devices, or a network. For more than one device, parallel or sequential division of processing may be used. Different devices making up the processor 1113 may perform different functions, such as extracting values for features by one device and applying a machine-learnt regression and mechanistic models by another device. In one embodiment, the processor 1113 is a control processor or other processor of the medical imaging system 1111. The processor 1113 operates pursuant to stored instructions to perform various acts described herein.

The processor 1113 is configured to extract values for features, to input the values, to output results, and/or to derive information from output results. The processor 1113 applies the machine-learnt model to data for one or more patients. The diagnosis, prognosis, therapy response, and/or other information is determined by the processor 1113 for a tumor or tumors of a patient.

The display 1116 is a CRT. LCD, plasma, projector, printer, or other output device for showing an image. The display 1116 displays the results or information derived from the results. Probabilities associated with any prediction. supporting data (e.g., values of input features), images from the medical scan data, and/or other information are output to assist the physician.

Turning now to FIG. 12, a computer system 1200 is generally shown in accordance with an aspect. The computer system 1200 can be an electronic computer framework comprising and/or employing any number and combination of computing devices and networks utilizing various communication technologies, as described herein. The computer system 1200 can be easily scalable, extensible, and modular, with the ability to change to different services or reconfigure some features independently of others. The computer system 1200 may be, for example, a server, desktop computer, laptop computer, tablet computer, or smartphone. In some examples, computer system 1200 may be a cloud computing node. Computer system 1200 may be described in the general context of computer-executable instructions, such as program modules, being executed by a computer system. Generally, program modules may include routines, programs, objects, components, logic, data structures, and so on that perform particular tasks or implement particular abstract data types. Computer system 1200 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 12, the computer system 1200 has one or more central processing units (CPU(s)) 1201a, 1201b, 1201c, etc. (collectively or generically referred to as processor(s) 1201). The processors 1201 can be a single-core processor, multi-core processor, computing cluster, or any number of other configurations. The processors 1201 can be any type of circuitry capable of executing instructions. The processors 1201, also referred to as processing circuits, are coupled via a system bus 1202 to a system memory 1203 and various other components. The system memory 1203 can include one or more memory devices, such as read-only memory (ROM) 1204 and a random-access memory (RAM) 1205. The ROM 1204 is coupled to the system bus 1202 and may include a basic input/output system (BIOS), which controls certain basic functions of the computer system 1200. The RAM is read-write memory coupled to the system bus 1202 for use by the processors 1201. The system memory 1203 provides temporary memory space for operations of said instructions during operation. The system memory 1203 can include random access memory (RAM), read-only memory, flash memory, or any other suitable memory systems.

The computer system 1200 comprises an input/output (I/O) adapter 1206 and a communications adapter 1207 coupled to the system bus 1202. The I/O adapter 1206 may be a small computer system interface (SCSI) adapter that communicates with a hard disk 1208 and/or any other similar component. The I/O adapter 1206 and the hard disk 1208 are collectively referred to herein as a mass storage 1210.

Software 1211 for execution on the computer system 1200 may be stored in the mass storage 1210. The mass storage 1210 is an example of a tangible storage medium readable by the processors 1201, where the software 1211 is stored as instructions for execution by the processors 1201 to cause the computer system 1200 to operate, such as is described hereinbelow with respect to the various Figures. Examples of computer program product and the execution of such instruction is discussed herein in more detail. The communications adapter 1207 interconnects the system bus 1202 with a network 1212, which may be an outside network, enabling the computer system 1200 to communicate with other such systems. In one aspect, a portion of the system memory 1203 and the mass storage 1210 collectively store an operating system, which may be any appropriate operating system to coordinate the functions of the various components shown in FIG. 12.

Additional input/output devices are shown as connected to the system bus 1202 via a display adapter 1215 and an interface adapter 1216. In one aspect, the adapters 1206, 1207, 1215, and 1216 may be connected to one or more I/O buses that are connected to the system bus 1202 via an intermediate bus bridge (not shown). A display 1219 (e.g., a screen or a display monitor) is connected to the system bus 1202 by a display adapter 1215, which may include a graphics controller to improve the performance of graphics-intensive applications and a video controller. A keyboard, a mouse, a touchscreen, one or more buttons, a speaker, etc., can be interconnected to the system bus 1202 via the interface adapter 1216, which may include, for example. a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 12, the computer system 1200 includes processing capability in the form of the processors 1201, and storage capability including the system memory 1203 and the mass storage 1210, input means such as the buttons, touchscreen, and output capability including the speaker 1223 and the display 1219.

In some aspects, the communications adapter 1207 can transmit data using any suitable interface or protocol, such as the internet small computer system interface, among others. The network 1212 may be a cellular network, a radio network, a wide area network (WAN), a local area network (LAN), or the Internet, among others. An external computing device may connect to the computer system 1200 through the network 1212. In some examples, an external computing device may be an external web server or a cloud computing node.

It is to be understood that the block diagram of FIG. 12 is not intended to indicate that the computer system 1200 is to include all of the components shown in FIG. 12. Rather, the computer system 1200 can include any appropriate fewer or additional components not illustrated in FIG. 12 (e.g., additional memory components, embedded controllers, modules, additional network interfaces, etc.). Further, the aspects described herein with respect to computer system 1200 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects. Various aspects can be combined to include two or more of the aspects described herein.

Aspects disclosed herein may be a system, a method, and/or a computer program product at any possible technical detail level of integration. The computer program product may include a computer-readable storage medium (or media) having computer-readable program instructions thereon for causing a processor to carry out various aspects.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine-dependent instructions, microcode, firmware instructions, state-setting data. configuration data for integrated circuitry, or either source-code or object code written in any combination of one or more programming languages, including an object-oriented programming language, such as Smalltalk, C++, high-level languages such as Python, or the like, and procedural programming languages, such as the “C” programming language or similar programming languages. The computer-readable program instructions may execute entirely on the user's computer, partly on the user's computer, as a stand-alone software package. partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry. in order to perform aspects of the present disclosure.

Aspects are described herein with reference to flowchart illustrations and/or block diagrams of methods. apparatus (systems), and computer program products according to aspects of the disclosure. It will be understood that each block of the flowchart illustrations and/or block diagrams, and combinations of blocks in the flowchart illustrations and/or block diagrams. can be implemented by computer-readable program instructions.

These computer-readable program instructions may be provided to a processor of a computer system, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer. other programmable data processing apparatus. or other device to cause a series of operational steps to be performed on the computer, other programmable apparatus or other devices to produce a computer-implemented process, such that the instructions which execute on the computer. other programmable apparatus, or other device implement the functions/acts specified in the flowchart and/or block diagram block or blocks.

The flowchart and block diagrams in the FIGS. Illustrate the architecture, functionality. and operation of possible implementations of systems. methods, and computer program products according to various aspects. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. Each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of. for example. layers. The terms “comprising”. “having”. “including”, and “containing” are to be construed as open-ended terms (i.e., meaning “including. but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

We claim:

1. A personalized method of treating a cancer patient with a tumor utilizing a computing system comprising a processing system and a memory system storing instructions that are executed by the processing system, the method comprising the computing system performing steps of:
performing parameter estimation to determine physiological parameters π of the tumor, including vascular hydraulic conductivity and interstitial hydraulic conductivity;
determining whether the selected tumor transport model is valid or invalid by solving for physiological parameters π, and
upon determining that the selected tumor transport model is valid, the method includes determining a treatment; and
the method further includes:
applying the treatment to a cancer patient.

2. The method of claim 1, wherein:
the step of performing parameter estimation to determine physiological parameters π includes:
measuring Peff and utilizing a parameter estimation problem to predict/determine K and L, where Peff is an effective permeability quantified as a rate of fluorescent signal passing through tumor vessel walls; Lp is a hydraulic conductivity of a microvascular wall (cm/mm Hg-sec); K is a hydraulic conductivity of tumor interstitium (cm2/mmHg-sec); or
measuring K directly and utilizing the experimental data when solving the parameter estimation problem.

3. The method of claim 2, wherein
the vascular density S/V is measured when measuring Peff vascular density S/V is measured when measuring Peff, where S/V is measured as vascular surface area per unit volume (cm−1).

4. The method of claim 3, wherein
the step of performing parameter estimation includes:
determining a time-dependent spatially-averaged drug concentration profile c_{avg}^{data}, representing a state of a tumor, including an ability of drugs and nutrients to accumulate in the tumor.

5. The method of claim 4, wherein the step of performing parameter estimation includes:
determining the time-dependent spatially-averaged drug concentration profile c_{avg}^{data} by utilizing:
$$\frac{{dc}_{avg}^{data}}{d t} = {P}_{eff} \frac{S}{V} ( {c}_{v} - {c}_{avg}^{data} )$$
where cᵥ is a solute concentration in vessels of a tumor (g/mL) and S/V is a vascular surface area per unit volume (cm⁻¹).

6. The method of claim 5, wherein the step of performing parameter estimation includes:
determining the physiological parameters π via a first parameter estimation problem:
$${min}_{π ∈ \prod } {\sum }_{i = 1}^{n} {( {\hat{c}}_{avg} ( {t}_{i} , π , {d}_{m} ) - {\hat{c}}_{avg}^{{}_{data}} ( {t}_{i} ) )}^{2} \\ s . t . {\hat{p}}_{peri} ( π ) \leq {\hat{p}}_{peri , max} \\ {\hat{p}}_{peri} ( π ) \geq {\hat{p}}_{peri , min} ,$$
where ĉ_{avg} is a dimensionless spatially-averaged concentration of solute that is determined by averaging a dimensionless concentration ĉ for all spatial nodes from a mechanistic solute transport model that is utilized as a parametric model output;
π=(Lₚ, K)∈Π⊂ [symbol] ⁿ^{_{π}} is a vector of physiological parameters of the spatially-averaged solute transport model; Lp being a hydraulic conductivity of a microvascular wall (cm/mm Hg-sec);
K is a hydraulic conductivity of tumor interstitium (cm2/mmHg-sec); and
dₘ is a diameter of a nanoscale (nm) biomolecule or macromolecular medicine.

7. The method of claim 6, wherein
upon determining that the model is invalid for this dataset, the method includes the computing system;
obtaining more data and solving the parameter estimation problem again; or
selecting a different tumor transport model, or modifying the tumor transport model, and solving the parameter estimation problem.

8. The method of claim 7, wherein utilizing the experimental data to model spatial-temporal transport in tumors includes the computing system performing the steps of:
utilizing a mechanistic tumor transport model directly; or
utilizing the mechanistic tumor transport model to generate simulation data to train a machine learning model.

9. The method of claim 8, wherein when repeating the step of performing parameter estimation, computing system performs the step of:
utilizes the first parameter estimation problem when determining the physiological parameters π with the mechanistic tumor transport model; or
utilizes a second parameter estimation problem when determining the physiological parameters π with the data-driven tumor transport model, the second parameter estimation problem being:
$${min}_{π ∈ \prod } {\sum }_{i = 1}^{n} {( {\hat{c}}_{avg , i}^{{}_{ANN}} ( π ) - {\hat{c}}_{avg}^{{}_{data}} ( {t}_{i} ) )}^{2} \\ s . t . {\hat{p}}_{peri} ( π ) \leq {\hat{p}}_{peri , max} \\ {\hat{p}}_{peri} ( π ) \geq {\hat{p}}_{peri , min} .$$
where ĉ^{ANN}_{avg} represents a dimensionless spatial average nanocarrier concentration at discrete time node i calculated from an ANN surrogate model, and
utilizing the experimental data when solving the parameter estimation problems.

10. The method of claim 9, wherein applying the treatment includes the computing system performing steps of:
determining a dose selection if the patient has not yet received adjunct therapy, where dose selection refers to the TME-normalizing agent, which is an adjunct; or
determining a drug-size selection if the patient has received adjunct therapy, where the drug-size selection refers to the anticancer drug, which is differentiated from the adjunct, which is the TME-normalizing agent.

11. The method of claim 10, wherein determining the dose selection includes the computing system performing steps of:
determining vascular hydraulic conductivity Lₚ and interstitial hydraulic conductivity K from empirical correlations between a cause-and-effect relationship between a tumor-normalizing dose, K and Lp; and
determining an optimal dose that maximizes a drug accumulation in tumors via determining:
$${max}_{x ∈ X} {\hat{c}}_{avg}^{} ( {t}_{f} , ( {f}_{{L}_{p}}^{j} ( x ) , {f}_{K}^{j} ( x ) ) , {d}_{m} )$$
with j∈{r, p}, where t_{f} is a final time, x is a TME-normalization regimen dose, and f_{Lp} and fₖ^{j} represent Lₚ and K, respectively, following treatment with the regimen dose, obtained from the experimental data.

12. The method of claim 11, wherein executing the drug-size includes the computing system performing step of:
determining an optimal size dₘ of the anticancer nanocarrier by determining
$${max}_{{d}_{m} ∈ Z} {\hat{c}}_{avg} ( {t}_{f} , π , {d}_{m} ) \\ s . t . {\hat{c}}_{peri} ( {t}_{f} , π , {d}_{m} ) \leq {λ}_{1} \\ {\hat{c}}_{peri} ( {t}_{f} , π , {d}_{m} ) \geq {λ}_{2} .$$
where λ₁ is a threshold for a safety constraint and λ₂ is a performance constraint.

13. The method of claim 10, wherein
determining the treatment includes the computing system performing step of simultaneously determining the dose selection and the drug-size.

14. The method of claim 13, wherein
determining the treatment includes the computing system performing step of applying empirical correlations that relate tumor physiology to adjunct dose.

15. The method of claim 14, wherein
the machine learning model is utilized, and determining the treatment includes the computing system performing steps of:
determining vascular hydraulic conductivity Lₚ and interstitial hydraulic conductivity K from empirical correlations between a cause-and-effect relationship between a tumor-normalizing dose, K and Lp, and determining
$${max}_{x ∈ X , {d}_{m} ∈ Z} {\hat{c}}_{avg} ( {t}_{f} , ( {f}_{{L}_{p}}^{j} ( x ) , {f}_{K}^{j} ( x ) ) , {d}_{m} ) \\ s . t . {\hat{c}}_{peri} ( {t}_{f} , ( {f}_{{L}_{p}}^{j} ( x ) , {f}_{K}^{j} ( x ) ) , {d}_{m} ) \leq {λ}_{1} \\ {\hat{c}}_{peri} ( {t}_{f} , ( {f}_{{L}_{p}}^{j} ( x ) , {f}_{K}^{j} ( x ) ) , {d}_{m} ) \geq {λ}_{2} .$$
with j∈{r, p}, where t_{f} is a final time, x is a TME-normalization regimen dose, and f_{Lp}^{j} and fₖ^{j} represent Lₚ and K, respectively, following treatment with the dose, obtained from the experimental data, λ₁ is a threshold for a safety constraint and λ₂ is a performance constraint.

16. The method of claim 15, wherein
the ANN surrogate model is utilized, and determining the treatment includes the computing system performing steps of:
determining vascular hydraulic conductivity Lₚ and interstitial hydraulic conductivity K from empirical correlations between a cause-and-effect relationship between a tumor-normalizing dose, K and Lₚ, and determining
$${max}_{x ∈ X , {d}_{m} ∈ Z} {\hat{c}}_{avg}^{{}_{ANN}} ( ( {f}_{{L}_{p}}^{j} ( x ) , {f}_{K}^{j} ( x ) ) , {d}_{m} ) \\ s . t . {\hat{c}}_{peri}^{{}_{ANN}} ( ( {f}_{{L}_{p}}^{j} ( x ) , {f}_{K}^{j} ( x ) ) , {d}_{m} ) \leq {λ}_{1} \\ {\hat{c}}_{peri}^{{}_{ANN}} ( ( {f}_{{L}_{p}}^{j} ( x ) , {f}_{K}^{j} ( x ) ) , {d}_{m} ) \geq {λ}_{2} .$$
with j∈{r, p}, where t_{f} is a final time, x is a TME-normalization regimen dose, and f_{Lp}^{j} and fₖ^{j} represent Lₚ and K, respectively, following treatment with the dose, obtained from the experimental data, λ₁ is a threshold for a safety constraint and λ₂ is a performance constraint.

17. A computerized system comprising:
a processing system and a memory system storing instructions that are executed by the processing system such that the system is configured to performing steps of:
performing parameter estimation to determine physiological parameters π of the tumor, including vascular hydraulic conductivity and interstitial hydraulic conductivity;
determining whether the selected tumor transport model is valid or invalid by solving for physiological parameters π, and upon determining that the selected tumor transport model is valid, the method includes determining a treatment; and
the method further includes:
applying the treatment to a cancer patient.

18. A computer program product comprising a memory device having computer executable instructions stored thereon, which when executed by one or more processors cause the one or more processors to perform a plurality of operations comprising:
performing parameter estimation to determine physiological parameters π of the tumor, including vascular hydraulic conductivity and interstitial hydraulic conductivity;
determining whether the selected tumor transport model is valid or invalid by solving for physiological parameters π, and
upon determining that the selected tumor transport model is valid, the method includes determining a treatment; and
the method further includes:
applying the treatment to a cancer patient.
